(19) United States
(12) Patent Application Publication
(10) Pub. No.: US 2024/0207134 A1
(43) Pub. Date: Jun. 27, 2024

(54) Devices, Methods, and Systems for Gait Modification

(71) Applicant: The trustees of Columbia University in the City of New York, New York, NY (US)

(72) Inventors: Sunil AGRAWAL, Newark, DE (US); Jesus Antonio PRADO DE LA MORA, New York, NY (US)

(73) Assignee: The trustees of Columbia University in the City of New York, New York, NY (US)

(21) Appl. No.: 18/603,989

(22) Filed: Mar. 13, 2024

Related U.S. Application Data
(63) Continuation of application No. PCT/US22/76338, filed on Sep. 13, 2022.
(60) Provisional application No. 63/246,072, filed on Sep. 20, 2021.

(51) Int. Cl. A61H 23/00

(52) U.S. Cl. CPC A61H 23/00; A61H 2201/5007; A61H 2201/5071; A61H 2205/106; A61H 2205/108; A61H 2230/625

(57) ABSTRACT

A quantitative gait modification and/or analysis system employs instrumented footwear, legwear, and an independent processing module. The instrumented footwear and/or legwear can have sensors that permit the extraction of gait kinematics in real time and provide feedback from it. Embodiments employing calibration-based estimation of kinematic gait parameters are described. An artificial neural network identifies gait stance phases in real-time and selective feedback is provided that targets certain portions of a subject's gait.

### CROSS REFERENCE TO RELATED APPLICATIONS

This application is a continuation of International Application PCT/US2022/076338, filed Sep. 13, 2022, which claims the benefit of U.S. Provisional Application 63/246,072, filed Sep. 20, 2021, each of which is incorporated herein by reference in its entirety.

### FIELD

The present disclosure relates generally to systems, methods, and devices for gait modification, and, more particularly, to a wearable apparatus for quantitative analysis of a subject's gait and/or providing feedback for gait modification of the subject.

### BACKGROUND

Patients, athletes, and other subjects could derive substantial benefits from gait modification. Camera-based gait analysis may provide a quantitative picture of gait issues. However, camera-based motion capture systems are expensive and are not available at many clinics. Auditory and tactile cueing (e.g., metronome beats and tapping of different parts of the body) are often used by physiotherapists to regulate subjects' gait and posture. However, this approach requires the practitioner to closely follow the subject and does not allow subjects to exercise on their own, outside the laboratory setting.

### SUMMARY OF THE INVENTION

Objects and advantages of embodiments of the disclosed subject matter will become apparent from the following description when considered in conjunction with the accompanying drawings.

One aspect of the invention is directed to a first apparatus for modifying a subject's gait. The first apparatus comprises a plurality of first transducers, a plurality of second transducers, and a processor. Each of the first transducers is configured for positioning proximate to a respective muscle of a right leg of the subject, and each of the second transducers is configured for positioning proximate to a respective muscle of a left leg of the subject. The processor is configured to track the subject's gait cycle and, based on the tracked gait cycle, actuate each of the plurality of first transducer and each of the plurality of second transducers at respective portions of the subject's gait cycle over multiple gait cycles.

In some embodiments of the first apparatus, the processor is configured so that each of the respective portions is synchronized to a respective time during which a respective corresponding muscle has its highest activation.

In some embodiments of the first apparatus, the plurality of first transducers includes a right tibialis anterior transducer, a right rectus femoris transducer, and a right biceps femoris transducer, and the plurality of second transducers includes a left tibialis anterior transducer, a left rectus femoris transducer, and a left biceps femoris transducer.

In some embodiments of the first apparatus, the plurality of first transducers includes a right tibialis anterior transducer, a right rectus femoris transducer, and a right biceps femoris transducer, and the plurality of second transducers includes a left tibialis anterior transducer, a left rectus femoris transducer, and a left biceps femoris transducer. In these embodiments, the processor is configured to (a) actuate the right tibialis anterior transducer during a first portion of the gait cycle, starting from right heel strike, (b) actuate the right rectus femoris transducer during a second portion of the gait cycle, (c) actuate the left biceps femoris transducer during a third portion of the gait cycle, (d) actuate the left tibialis anterior transducer during a fourth portion of the gait cycle, (c) actuate the left rectus femoris transducer during a fifth portion of the gait cycle, (f) actuate the right biceps femoris transducer during a sixth portion of the gait cycle. Optionally, in these embodiments, the first, second, third, fourth, fifth, and sixth portions of the gait cycle are all non-overlapping.

In some embodiments of the first apparatus, the plurality of first transducers includes a right tibialis anterior transducer, a right rectus femoris transducer, and a right biceps femoris transducer, and the plurality of second transducers includes a left tibialis anterior transducer, a left rectus femoris transducer, and a left biceps femoris transducer. In these embodiments, the processor is configured to (a) actuate the right tibialis anterior transducer from 0-10% of the gait cycle, starting from right heel strike, (b) actuate the right rectus femoris transducer from 10-30% of the gait cycle, (c) actuate the left biceps femoris transducer from 30-40% of the gait cycle, (d) actuate the left tibialis anterior transducer from 40-60% of the gait cycle, (e) actuate the left rectus femoris transducer from 60-75% of the gait cycle, and (f) actuate the right biceps femoris transducer from 87-100% of the gait cycle.

In some embodiments of the first apparatus, each of the first transducers and each of the second transducers comprises a vibrator configured to couple vibration into a respective leg muscle.

Some embodiments of the first apparatus further comprise at least two pressure sensors configured for positioning at the subject's shoe. In these embodiments, the processor is configured to track the subject's gait cycle based on signals received from the at least two pressure sensors. And optionally in these embodiments, the subject's gait cycle is tracked using a trained machine learning model that receives signals from the at least two pressure sensors as input.

Another aspect of the invention is directed to a first method for modifying a subject's gait. The first method comprises positioning each of a plurality of first transducers proximate to a respective muscle of a right leg of the subject, and positioning each of a plurality of second transducers proximate to a respective muscle of a left leg of the subject. The first method also comprises tracking the subject's gait cycle, and actuating each of the plurality of first transducer and each of the plurality of second transducers at respective portions of the subject's gait cycle over multiple gait cycles based on the tracked gait cycle.

In some instances of the first method, each of the respective portions is synchronized to a respective time during which a respective corresponding muscle has its highest activation.

In some instances of the first method, the plurality of first transducers includes a right tibialis anterior transducer, a right rectus femoris transducer, and a right biceps femoris transducer, and the plurality of second transducers includes a left tibialis anterior transducer, a left rectus femoris transducer, and a left biceps femoris transducer.

In some instances of the first method, the plurality of first transducers includes a right tibialis anterior transducer, a right rectus femoris transducer, and a right biceps femoris transducer, and the plurality of second transducers includes a left tibialis anterior transducer, a left rectus femoris transducer, and a left biceps femoris transducer. And the actuating comprises (a) actuating the right tibialis anterior transducer during a first portion of the gait cycle, starting from right heel strike, (b) actuating the right rectus femoris transducer during a second portion of the gait cycle, (c) actuating the left biceps femoris transducer during a third portion of the gait cycle, (d) actuating the left tibialis anterior transducer during a fourth portion of the gait cycle, (c) actuating the left rectus femoris transducer during a fifth portion of the gait cycle, (f) actuating the right biceps femoris transducer during a sixth portion of the gait cycle. Optionally, in these instances, the first, second, third, fourth, fifth, and sixth portions of the gait cycle are all non-overlapping.

In some instances of the first method, the plurality of first transducers includes a right tibialis anterior transducer, a right rectus femoris transducer, and a right biceps femoris transducer, and the plurality of second transducers includes a left tibialis anterior transducer, a left rectus femoris transducer, and a left biceps femoris transducer. And the actuating comprises (a) actuating the right tibialis anterior transducer from 0-10% of the gait cycle, starting from right heel strike, (b) actuating the right rectus femoris transducer from 10-30% of the gait cycle, (c) actuating the left biceps femoris transducer from 30-40% of the gait cycle, (d) actuating the left tibialis anterior transducer from 40-60% of the gait cycle, (e) actuating the left rectus femoris transducer from 60-75% of the gait cycle, and (f) actuating the right biceps femoris transducer from 87-100% of the gait cycle.

In some instances of the first method, each of the first transducers and each of the second transducers comprises a vibrator configured to couple vibration into a respective leg muscle.

In some instances of the first method, the tracking of the subject's gait cycle is based on signals received from the at least two pressure sensors. Optionally, in these instances, the subject's gait cycle is tracked using a trained machine learning model that receives signals from the at least two pressure sensors as input.

Another aspect of the invention is direct to a second apparatus for modifying a subject's gait. The second apparatus comprises at least two pressure sensors and a first transducer configured for positioning proximate to a first leg muscle of the subject. The second apparatus also comprises a processor configured to track the subject's gait cycle responsively to signals from the at least two pressure sensors and, based on the tracked gait cycle, actuate the first transducer to provide feedback to the subject in synchronicity with a first portion of the subject's gait cycle.

In some embodiments of the second apparatus, the processor is configured to actuate the first transducer in synchronicity with the first portion of the subject's gait cycle over multiple gait cycles. In some embodiments of the second apparatus, the first transducer comprises a vibrator configured to couple vibration into the first leg muscle.

In some embodiments of the second apparatus, the subject's gait cycle is tracked using a trained machine learning model that receives signals from the at least two pressure sensors as input.

Some embodiments of the second apparatus further comprise a second transducer configured for positioning proximate to a second leg muscle of the subject, and the processor is configured to actuate the second transducer to provide feedback to the subject in synchronicity with a second portion of the subject's gait cycle.

In some embodiments of the second apparatus, the processor is configured to actuate the first transducer in synchronicity with the first portion of the subject's gait cycle and the second transducer in synchronicity with the second portion of the subject's gait cycle over multiple gait cycles. In some embodiments of the second apparatus, the first portion of the subject's gait cycle does not overlap with the second portion of the subject's gait cycle.

In some embodiments of the second apparatus, the feedback comprises haptic feedback at a frequency between 60 and 100 Hz. In some embodiments of the second apparatus, the at least two pressure sensors are configured for positioning at the subject's shoe. In some embodiments of the second apparatus, the apparatus comprises an accelerometer, and the processor is configured to track the subject's gait cycle responsively to signals from the at least two pressure sensors and the accelerometer.

In some embodiments of the second apparatus, the processor is configured to actuate the first transducer upon detection of a change in stride characteristic of the tracked gait cycle. In some embodiments of the second apparatus, the processor is configured to actuate the first transducer upon detection of a change in stride length of the tracked gait cycle.

Another aspect of the invention is direct to a second method for modifying a subject's gait. The second method comprises positioning a first transducer proximate to a first leg muscle of a subject, tracking the subject's gait cycle, and based on the tracked gait cycle, providing feedback to the first leg muscle by actuating the first transducer in synchronicity with a first portion of the subject's gait cycle.

In some instances of the second method, the actuating of the first transducer in synchronicity with the first portion of the subject's gait cycle causes a modification to the subject's gait. In some instances of the second method, the first transducer is actuated in synchronicity with the first portion of the subject's gait cycle over multiple gait cycles.

In some instances of the second method, the subject's gait cycle is tracked using a trained machine learning model. In some instances of the second method, the trained machine learning model is configured to track the subject's gait using signals received from a set of sensors over multiple gait cycles, and the set of sensors comprises one of a) two pressure sensors, or b) a pressure sensor and an accelerometer.

Some instances of the second method further comprise positioning a second transducer proximate to a second leg muscle of a subject, and, based on the tracked gait cycle, providing feedback to the second leg muscle by actuating the second transducer in synchronicity with a second portion of the subject's gait cycle.

In some instances of the second method, the first portion of the subject's gait cycle does not overlap with the second portion of the subject's gait cycle. In some instances of the second method, at least some of the first portion of the subject's gait cycle overlaps with the second portion of the subject's gait cycle. In some instances of the second method, the first transducer is actuated in synchronicity with the first portion of the subject's gait cycle and the second transducer is actuated in synchronicity with the second portion of the subject's gait cycle over multiple gait cycles.

In some instances of the second method, the first transducer is actuated upon detection of a change in stride characteristic of the tracked gait cycle. In some instances of the second method, the first transducer is actuated upon detection of a change in stride length of the tracked gait cycle.

Another aspect of the invention is directed to a third apparatus for detecting freezing of gait (FOG) events for a subject. The third apparatus comprises a first plurality of pressure sensors, each of which is configured to detect a pressure at a respective position beneath the subject's right foot, and a second plurality of pressure sensors, each of which is configured to detect a pressure at a respective position beneath the subject's left foot. The third apparatus also comprises an artificial neural network configured to detect FOG events based on the pressures detected by the first plurality of pressure sensors and the pressures detected by the second plurality of pressure sensors.

Some embodiments of the third apparatus further comprise a transducer configured for positioning against a leg muscle of the subject. The transducer is actuated in response to detection of an FOG event by the artificial neural network. Optionally, in these embodiments, the transducer can comprise a vibrator configured to couple vibration into the leg muscle.

Another aspect of the invention is directed to a third method of detecting freezing of gait (FOG) events for a subject. The third method comprises detecting a pressure at each of a plurality of first positions beneath the subject's right foot, and detecting a pressure at each of a plurality of second positions beneath the subject's left foot. The third method also comprises processing the pressure at each of the plurality of first positions and the pressure at each of the plurality of second positions using an artificial neural network configured to detect FOG events based on the pressure at each of the plurality of first positions and the pressure at each of the plurality of second positions.

Some instances of the third method further comprise actuating a transducer positioned against a leg muscle of the subject in response to detection of an FOG event. Optionally, in these instances, the transducer comprises a vibrator configured to couple vibration into the leg muscle.

### BRIEF DESCRIPTION OF THE DRAWINGS

Embodiments will hereinafter be described with reference to the accompanying drawings, which have not necessarily been drawn to scale. Where applicable, some features may not be illustrated to assist in the illustration and description of underlying features. Throughout the figures, like reference numerals denote like elements.

FIG. 1 is a schematic diagram illustrating components of a system for gait analysis and training, according to one or more embodiments of the disclosed subject matter.

FIG. 2A is a schematic diagram illustrating components of a footwear unit of a system for gait analysis and training, according to one or more embodiments of the disclosed subject matter.

FIGS. 2B and 2C are side and bottom views of an exemplary footwear module for gait analysis and training, according to one or more embodiments of the disclosed subject matter.

FIG. 3 illustrates a DeepSole system, according to one or more embodiments of the disclosed subject matter.

FIG. 4 depicts a printed circuit board according to an embodiment of the DeepSole system.

FIG. 5 illustrates an overview of the gait percentage timed vibration, according to one or more embodiments of the disclosed subject matter.

FIG. 6 illustrates a diagram with gait cycle percentage synchronized haptic feedback on muscle groups, according to one or more embodiments of the disclosed subject matter.

FIG. 7 illustrates the muscle targeted by the synchronized vibration, according to one or more embodiments of the disclosed subject matter.

FIG. 8 is a graphical representation of gait events in a typical gait cycle.

FIGS. 9A and 9B illustrate box plots of the gait parameters for constant vibration, according to one or more embodiments of the disclosed subject matter.

FIGS. 10A and 10B illustrate box plots of the gait parameters for timed vibration, according to one or more embodiments of the disclosed subject matter.

FIG. 11 illustrates box plots for the spatial parameters of ten PD patients, according to one or more embodiments of the disclosed subject matter.

FIG. 12 illustrates box plots for the balance parameters of ten PD patients, according to one or more embodiments of the disclosed subject matter.

FIG. 13 illustrates box plots for the temporal parameters of ten PD patients, according to one or more embodiments of the disclosed subject matter.

FIG. 14 illustrates and overview of a neural network with an Encoder-Decoder RNN architecture, according to one or more embodiments of the disclosed subject matter.

FIG. 15 illustrates bar plots of the four metrics used to evaluate a model, according to one or more embodiments of the disclosed subject matter.

### DETAILED DESCRIPTION OF THE PREFERRED EMBODIMENTS

### Section I

This application describes advances that go beyond the disclosure of patent application US 2020/0000373, which is incorporated herein by reference in its entirety.

Embodiments implement feedback to a subject during selective portions of the subject's gait cycle. For example, vibratory feedback can be provided at the select portions to modify the subject's gait. Movement involves the coordination of different muscle groups. To successfully perform motions, mechanosensors provide proprioceptive information to know the current state of the joints and limb segments. The motor system activates the muscles accordingly to coordinate the joints according to movement goals. Vibratory feedback can enhance the information sensed by the mechanosensors and affect the closed-loop locomotion control, thereby affecting the gait of a human user.

Embodiments include two types of vibratory inputs at six different muscles: constant vibration of the muscles during the gait cycle and timed vibration synchronized to the percentage of the gait cycle when they are active. The results show that both vibratory strategies have an effect on the gait of healthy subjects. Modification to the subjects' stride velocity and cadence was observed. Gait characteristics depend both on spatial and temporal parameters. In some implementations, it was observed that constant vibration modified the spatial parameters, while timed vibration modified the temporal parameters.

In one or more embodiments, a gait analysis and training system can be used to target or select portions of the subject's gait for feedback. Embodiments can provide subjects with auditory and/or vibrotactile feedback that is automatically generated by software in real-time, with the aim of modifying their movements. The gait analysis and training system can be a wearable gait analysis and sensory feedback device targeted for subjects. As the subject walks, the system can measure underfoot pressure, ankle motion, feet movement and generate data that can correspond to motion dynamics and, responsively to these data, generate preselected feedback with the aim of modifying gait patterns.

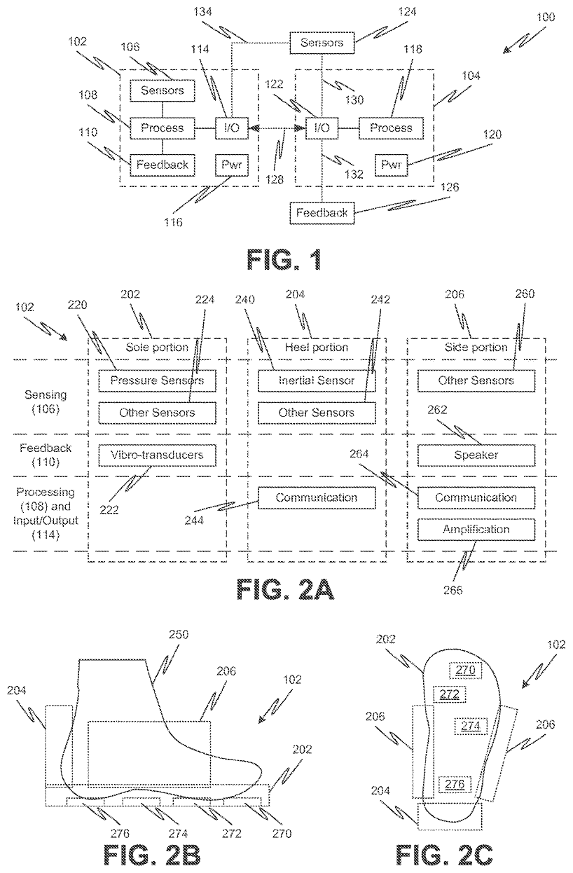

Referring to FIG. 1, a gait analysis and training system 100 can include one or more footwear modules 102 and a wearable processing module 104. The footwear unit 102 can include one or more sensors 106 that measure characteristics of the subject's gait as the subject walks, including underfoot pressure, acceleration, or other foot kinematics. The system can also include one or more remote sensors 124 disposed separate from the footwear unit 102, for example, on the shank or belt of the subject. Sensor signals from the remote sensors 124 can be communicated to the closest footwear module 102, for example, via a wired or wireless connection 134 for transmission to the remote processor 118 together with data from sensors 106 via connection 128. Alternatively, sensor signals from the remote sensors 124 can be communicated directly to the remote processor 118, for example, by wireless connection 130.

An on-board processing unit 108 can receive signals from the one or more sensors 106, 124 and prepare data responsively to the sensor signals for transmission to a remote processor 118 of the wearable processing module 104, for example, via transmission 128 between communication module 114 in the footwear unit 102 and a corresponding communication module 122 in the wearable processing module 104. The on-board processing unit 108 can include, for example, an analog to digital converter or microcontroller. For example, the transmission 128 of sensor data can be via wireless transmission

The remote processor 118 of the wearable processing module 104 can receive the sensor data and determine one or more gait parameters responsively thereto. The remote processor 118 can further provide feedback, such as vibratory or audio feedback, based on the sensor data and determined gait parameters, for example, to modify the subject's gait. For example, the feedback can be provided via one or more transducers 110 (e.g., in the footwear unit), such as vibrotactile transducers or speakers. The transmission 128 of feedback signals from the processor 118 to the feedback transducers 110 can be via a wired connection, such as audio cables. Alternatively or additionally, the feedback can be provided via one or more remote feedback modules 126 via a wired or wireless connection 132. For example, the remote feedback module 126 can provide audio feedback via headphones worn by the subject, audio feedback via a speaker worn by the subject, tactile feedback via transducers mounted on the body of the subject remote from the foot, or visual feedback via one or more flashing lights.

The wearable processing module 104 can include an independent power supply 120, such as a battery, that provides electrical power to the components of the processing module 104, e.g., the remote processor 118 and the communication module 122. In addition, each footwear module 102 can include an independent power supply 116, such as a battery, that provides electrical power to the components of the footwear unit 102, e.g., the sensors 106, the on-board processing unit 108, the feedback transducers 110, and the communication module 114. Alternatively or additionally, the power supply 120 of the wearable processing module 104 can supply power to both the processing module 104 and the footwear units 102, for example, via one or more cables connecting the processing module 104 to each footwear module 102.

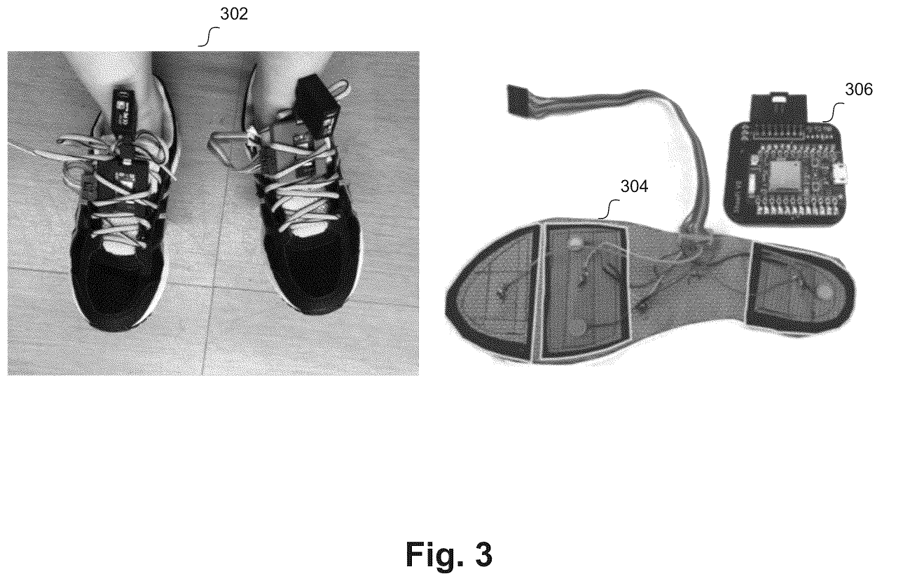

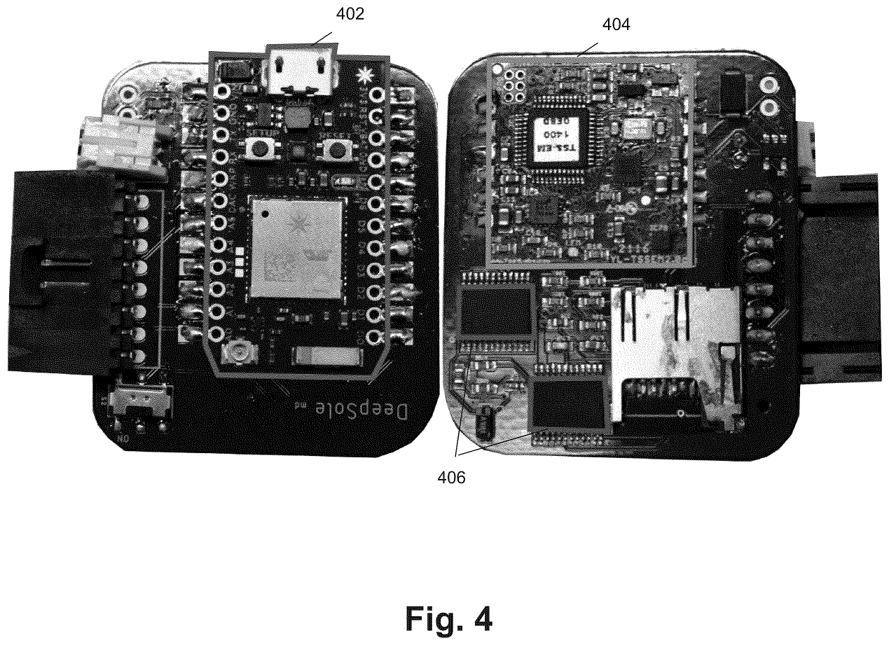

Each footwear module 102 can include at least a sole portion 202, a heel portion 204, and one or more side portions 206, as illustrated in FIGS. 2A-2C. For example, each portion of the footwear unit 102 can include sensing portions 106, feedback portions 110, and processing 108 or communication 114 portions. The sole portion 202 can include one or more pressure sensors 220 as part of sensing portion 106. Optionally, the sole portion 202 can further include one or more other sensors 224, such as an inertial measurement unit. The sole portion 202 can further include one or more vibrotactile transducers 222 as part of the feedback portion 110. The heel portion 204 of the footwear unit 102 can include one or more inertial sensors 240, such as an inertial measurement unit. Optionally, the heel portion 204 can further include one or more other sensors 242, such as an accelerometer. The heel portion 204 can further include a communication module 244, for example, a wireless communication module to transmit data from sensing portions 106 of the heel portion 204 and/or the sole portion 202. The side portions 206 can optionally include one or more other sensors, such as an ultrasonic base sensor, as part of sensing portion 106. The side portions 206 can further include a speaker 262 as part of the feedback portion 110 and a communication module 264, for example, a wired communication module to transmit feedback signals from a remote processor to the speaker 262 and/or the vibrotactile transducers 222 of the sole portion. The side portions 206 can also include an amplification module 266 to amplify the feedback signals from the remote processor.

As illustrated in FIGS. 2B-2C, feedback components and sensing devices in the sole portion 202 of the footwear unit 102 can be grouped together at various regions 270-276 along the bottom of the foot 250. For example, each region 270-276 can include at least one feedback transducer (e.g., a vibro-transducer) and at least one pressure sensor (e.g., a piezo-resistive sensor). Feedback/sensing region 270 can be disposed under the hallux distal phalanx. Feedback/sensing region 272 can be disposed under the first metatarsal head. Feedback/sensing region 274 can be disposed under the middle lateral arch and/or the fourth metatarsal head. Feedback/sensing region 276 can be disposed under the calcancous.

Human movement is complex, as even simple motion requires coordination of several muscle groups. Furthermore, execution of the motion requires closed-loop control between the sensors and the muscles as actuators. This loop control involves a good sense of the position and movement of all parts of the body, also known as kinesthesia and proprioception. Proprioception relies on mechanosensors, also known as proprioceptors, distributed throughout the body. During movement, the mechanosensors register the changes in the tissues throughout the motion. This includes muscle length and tension, deformation of tissues like tendons, joint capsules, ligaments, and skin. The information from the proprioceptors is constantly monitored by the central or peripheral nervous system. Muscle spindles are used to sense the state of the muscles. These receptors are located in parallel with the muscle fibers and signal the length changes. Studies have found that the sense of position and the sense of motion are processed separately within the central nervous system. The muscle spindles provide two information channels that fire at different rates when the muscle is being stretched or held under load. The signal from the spindles change depending on the muscle's previous history of length and contraction changes.

Golgi tendons and skin receptors also contribute to the proprioception. The tendons are in series with the muscles and can detect the tension when a load is applied. When the muscle activates, there is an initial signal peak followed by a plateau. The signal's amplitude depends on the rate of change of the tension and its absolute maximum value. When the muscles and tendons are subjected to continuous vibration, the muscle spindles give the illusion of limb movement. This was demonstrated by Goodwin et al. by providing vibration at the elbow level to subjects while blindfolded. The vibration was applied only to one side, and the subjects were asked to track the position with their other arm. The experimenters reported that there was a lag in the arm tracking and a persistent error in the tracking arm. Although, when the vibration is presented both at the agonist and antagonist muscle groups, it does not create the illusion of movement.

Furthermore, vibratory stimuli have different effects on muscles that are relaxed than those that are contracted. For example, high-frequency vibration can enhance movement speed by increasing preparatory beta synchronization and placing the motor cortex in a “ready-to-move” state. This is because the vibration can decouple the tension and length information. Macerollo et al. measured EEG signals in a sample of healthy subjects before, during, and after peripheral surface vibration while subjects were at rest. The data revealed a significant decrease in beta power (15-30 Hz) over the contralateral sensorimotor cortex at the onset and offset of 80 Hz vibrations. This vibration can modulate the uncertainty of the proprioceptive afferent signal, improving motor performance by stabilizing proximal joints and improving the stability of the complete motion.

In quiet standing, a vibration on the tibialis anterior elicits a forward body tilt, whereas vibrations of the hamstring and triceps surae elicit a backward trunk tilt. In treadmill locomotion, hamstring vibrations produce forward stepping. Several muscles have been studied, and it has been shown that the surface vibration has a significant effect on the area where the vibration is applied during locomotion. During treadmill walking, when a constant vibration is applied to the hamstrings, subjects significantly increase their walking velocity and cadence.

Furthermore, when the vibration is different on the left and right side, the subjects' limbs are affected differently. This could make the subjects more or less stable. Due to the inherent complexity of continuous gait tracking during overground walking, gait timed vibration has not been adequately studied. Most of the examples present in the literature use specific gait events, such as heel strike to time the vibration. Duclos et al. provided vibratory feedback at the triceps surae during stance phase but found no statistical difference in the spatiotemporal parameters during the cycle. Roden-Reynolds et al. provided vibration to the gluteus medius during the swing phase. This strategy resulted in a wider stride width when the vibration was present. These studies used gait events to provide feedback, but complex synchronized feedback to the muscles during walking has not yet been explored. The accurate prediction of the gait cycle percentage in realtime opens the door to provide complex synchronized feedback to individuals during overground walking.

Furthermore, using the modular design architecture of embodiments of DeepSole, system functionality can be expanded with vibration motors. This feature can be used to provide continuous or intermittent haptic feedback throughout the gait cycle, with time synchronized vibration. This application shows that timed vibration on several muscle groups when they are being used during walking will modify the cadence and velocity of subjects, by exciting the muscle spindles. Furthermore, an analysis is presented on how different vibration strategies modify the gait of participating subjects.

Current systems for human motion estimation and tracking involve placement of numerous trackers over the body of a human user. These systems are either bound to a single room, e.g., motion tracking using cameras, or use wireless sensors, e.g., Inertial Measurement Units-based (IMU) systems, which can become unreliable during prolonged use. These sensor systems can track the human motion with different levels of accuracy but are incapable of providing sensory, or haptic, feedback to the user. Furthermore, the recorded data from the markers is often post-processed. This procedure can be time intensive and involve extensive human intervention to resolve conflicts due to poor quality of the data, lost data, or drift in the sensor measurement system due to the underlying technology. The post-processing time and its complexity increases with the number of markers and duration of the recorded session. In summary, movement data for a few minutes may involve hours of work. Furthermore, motion capture devices are expensive. Some full-body IMU systems may cost in the order of tens of thousand dollars and camera-based systems can be even more expensive.

These high prices of the sensory hardware greatly reduce access to these technologies in smaller medical centers. The costs are much higher, considering the number of man hours used to process the data and obtain meaningful information. Some of the embodiments described herein greatly reduce the cost of the hardware from thousands to only a few hundred dollars. This is possible by using a modular design with independent trackers. In some embodiments, each tracker module includes an IMU and a micro-controller board. For example, the board can be capable of recording 9 motion signals (3D acceleration, 3D angular velocity, and 3D orientation), and 3 analog sensors. Additionally, the board can be capable of independently controlling up to 3 direct current motors. In some embodiments, the module's communication with other modules and a host computer is wireless. In combination with this module, some embodiments can use machine learning to track the motion of the body segments of the wearer in real-time. In some embodiments, the module can provide sensory (haptic) feedback to the wearer at different points on the human body synchronized with the body motion. For example, the feedback can be provided using onboard motor drivers, or from an external device. In some embodiments, the motion information can be transmitted using the User Datagram Protocol (UDP) to another device. Several experiments have been conducted with the system to assess its capabilities to (i) characterize gait of human users in real time, (ii) provide localized leg muscle vibration timed to gait cycle percentage, and (iii) the effects of synchronized feedback on the gait of human users.

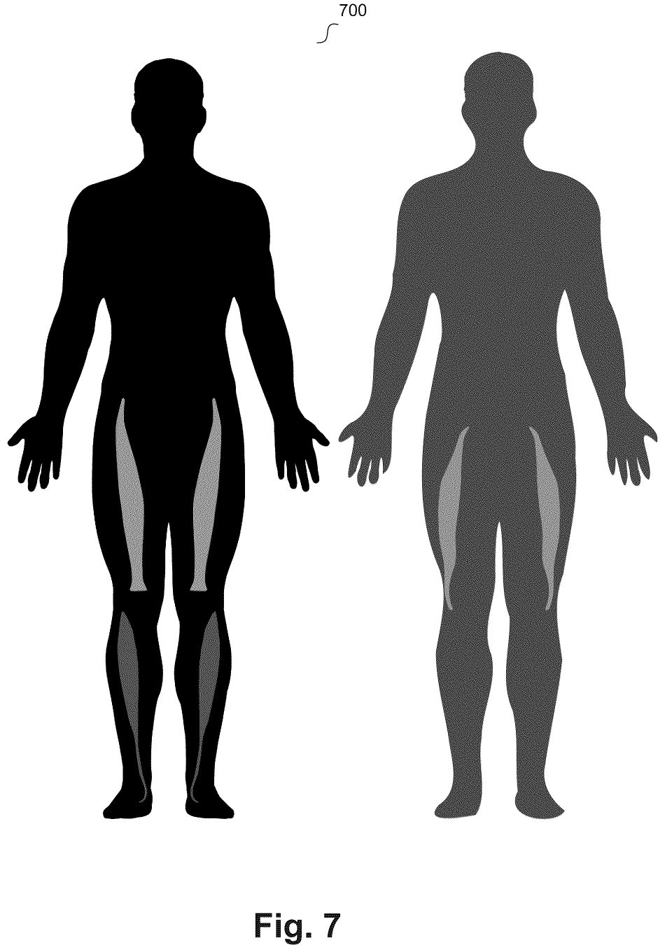

Experimental Design: A total of seven healthy subjects participated in an experiment. The subjects were naive to the experiment and agreed to participate by signing a consent form. The protocol was reviewed and approved by the Columbia University Institutional Review Board. The experiment included a total 39 minutes of walking divided into 2 conditions—FIG. 5 illustrates diagram 500 that depicts an overview of the gait percentage timed vibration protocol. During the experiment, data were collected by both the DeepSole system and the Zeno walkway.

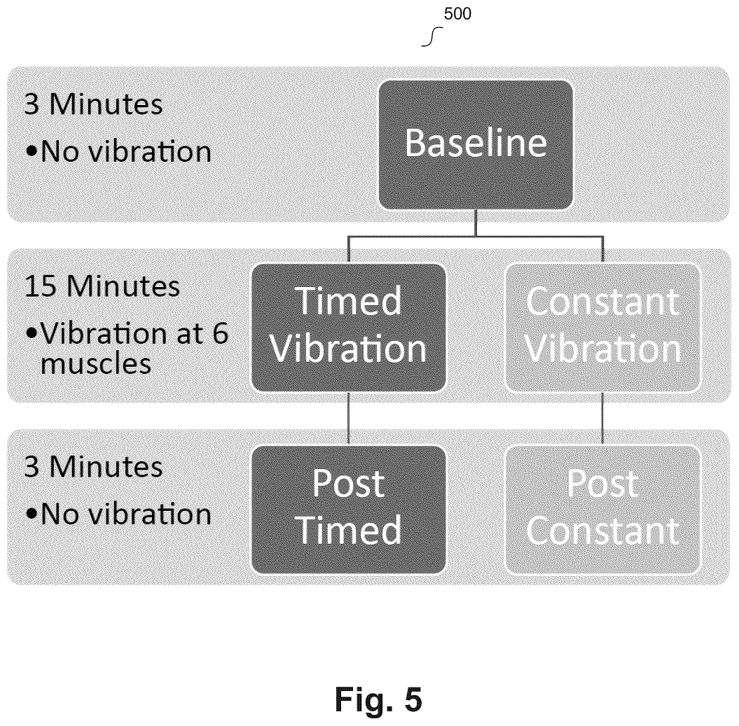

FIG. 3 illustrates the DeepSole system, a soft, flexible, instrumented insole which can be comfortably inserted into any standard footwear. Panel 302 illustrates a subject wearing the DeepSole system. Element 306 depicts a printed circuit board with microcontroller and IMU, and the instrumented insole 304 includes pressure sensors and vibration motors. The insoles are a custom pressure sensor made with piezo resistive e-textiles. They can measure pressure under the phalanges, the metatarsals, and the calcaneus.

By placing the sensors in the aforementioned locations, loading changes during the gait can be captured. In some embodiments, the system collects signals from twelve channels: three pressure signals, three linear accelerations, three angular velocities, and three Euler angles. The accelerations, angular velocity, and Euler angles can be measured in the local IMU coordinate system. In some embodiments, the sensor readings can be recorded (e.g., at 50 Hz on an on-board microSD card) and streamed (e.g., through WiFi using User Datagram Protocol (UDP) data packets) for logging and real-time prediction of the current gait cycle percentage.

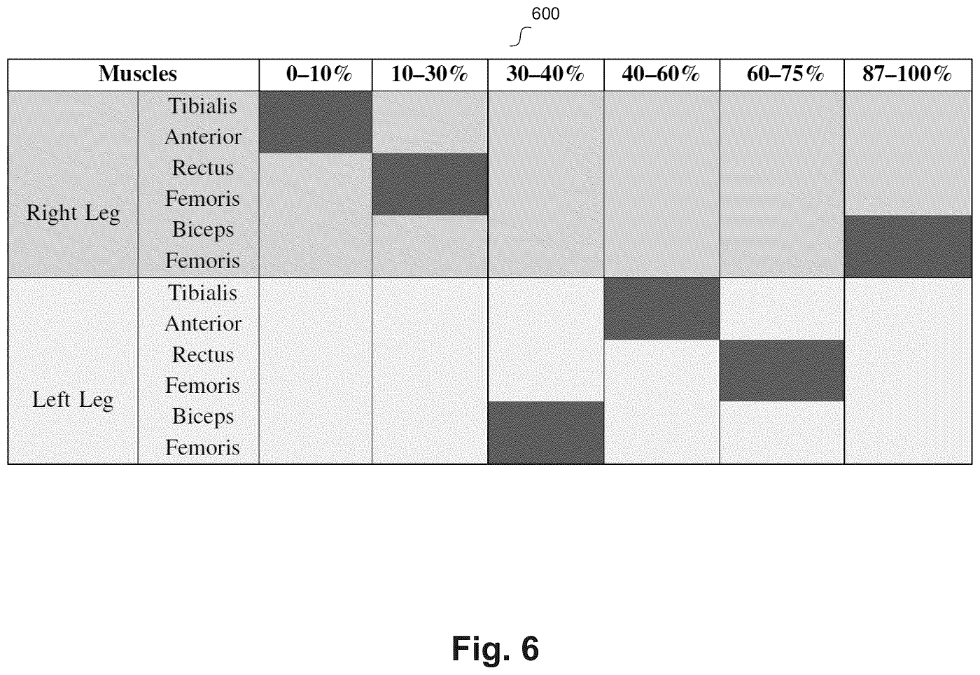

For the described experiment, embodiments of the DeepSole System included six units which were placed at: two units were mounted on the shoes, two mounted on the thighs, and two mounted on the shanks. The thigh units controlled two vibration motors each, mounted at the rectus femoris and biceps femoris of the corresponding side. The shank units controlled one vibrator each, mounted at the tibialis anterior. The leg units were secured to the subjects using a 3d printed case and Velcro straps, and the vibrators were positioned on the belly of the muscle along the direction of the muscle fibers and secured using medical tape. FIG. 4 depicts a printed circuit board according to an embodiment of the DeepSole system. A front view of the DeepSole board (Left) and rear view of the DeepSole board (Right) are depicted. Embodiments of the board include a microcontroller unit 402, IMU 404, and motor drivers 406.

For the baseline (BA), post constant (PC), and post timed (PT) sessions, the subjects walked at a self-selected speed for 3 minutes. For these data collection sessions, the DeepSole system was used for data collection without vibratory feedback. The order of the constant vibration (CV), and timed vibration (TV) sessions was counterbalanced. Four subjects experienced TV first and three subjects experienced CV first.

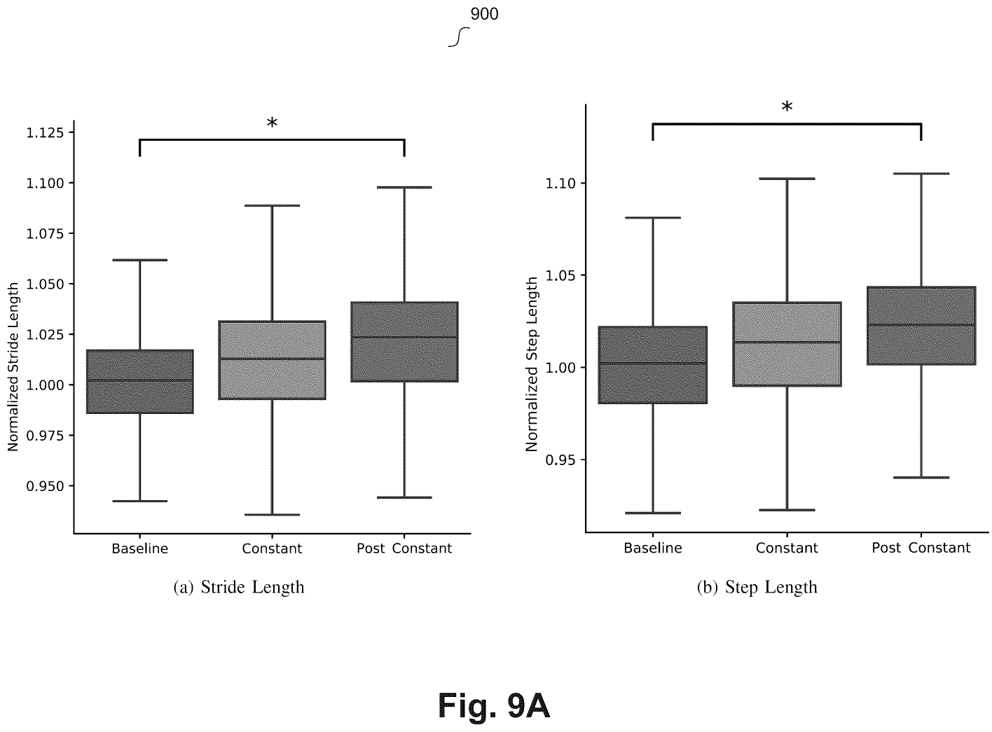

For the TV session, haptic stimulation was provided to several muscles based on the current gait cycle percentage. The subjects walked for several minutes wearing the DeepSole system enhanced with 6 vibrator motors placed at the right and left sides: tibialis anterior, rectus femoris, and biceps femoris. Subjects walked for 15 minutes and the data were streamed to a computer where the gait cycle percentage was calculated. The muscle targeted by the synchronized vibration are shown in FIG. 7. Diagram 702 includes a frontal view of a person and a back view. The muscle groups include the Tibialis anterior, Rectus femoris, and Biceps femoris. Other suitable locations for the vibrators and numbers of vibrators can be implemented.

The CV session included 15 minutes of walking. During this time, an embodiment of the DeepSole system streamed the sensor data to a computer. Six vibrators were turned on at a frequency of 80 Hz for the duration of the session. The 80 Hz frequency was selected as it has been shown to have an effect on muscle response. The subjects were asked to walk back and forth on the instrumented mat at a self-selected speed for the duration of the session.

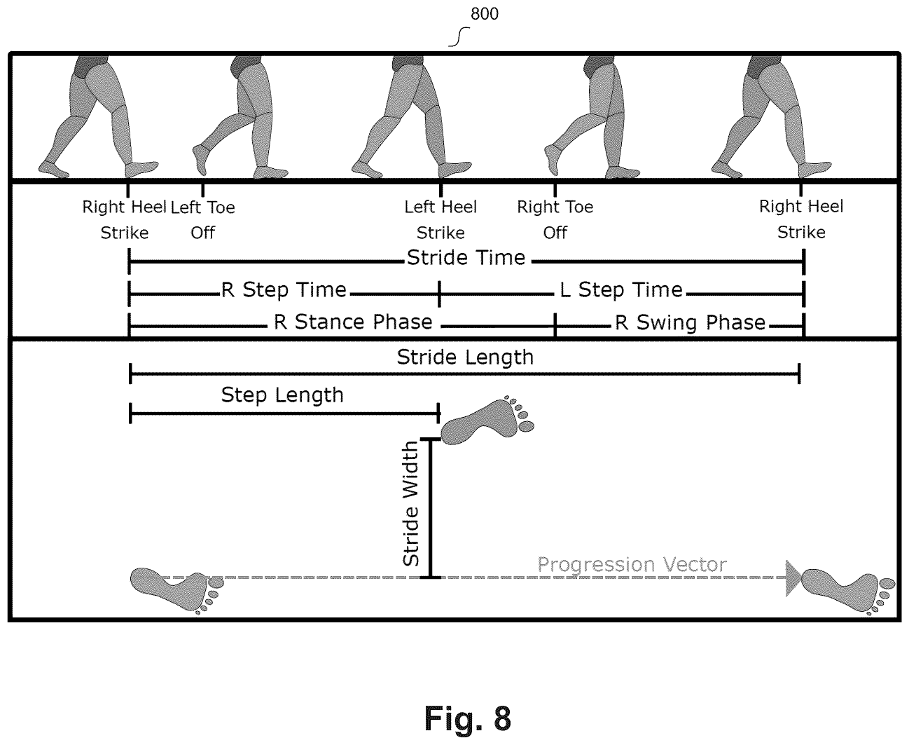

The timed vibration feedback activation during the TV session was a function of the gait cycle measured from the right leg of the subjects. From 0-10% of the gait cycle, starting from right heel strike, the right tibialis anterior was activated; 10-30% the right rectus femoris; from 30-40% the left biceps femoris; 40-60% the left tibialis anterior; 60-75% the left rectus femoris; and from 87-100% right biceps femoris. This pattern was repeated during the full TV session. The vibration activation is shown in FIG. 6 and table 600.

Gait Parameters Evaluated: To see the effects of the vibration feedback on the gait of the subjects, eight gait parameters were chosen. These parameters were obtained from the Zeno Walkway software (PKMAS). The strides used in this analysis are only the strides performed on the instrumented mat. Stride length (SL), stride width (SW), and step length (TL) were chosen to see the effects on the spatial characteristics of the gait. For the temporal characteristics, stance time (ST), swing time (WT), and stride time (TT) were chosen.

ST is the duration that the corresponding foot is in contact with the ground; this is between heel strike and toe off. WT is the duration between toe off to the subsequent heel strike of the same side, i.e., the period that the foot is not in contact with the floor. TT is the duration between heel strike to heel strike of the same side, which also corresponds to ST+WT. The progression vector is defined from the location of the heel strike from one foot to the subsequent heel strike of the same foot. The norm of this vector is the SL. SW is defined as the perpendicular distance from the progression vector to the heel strike of the opposite foot. TL is defined as the distance from the location of the heel strike to the heel strike of the opposite foot, measured along the progression vector.

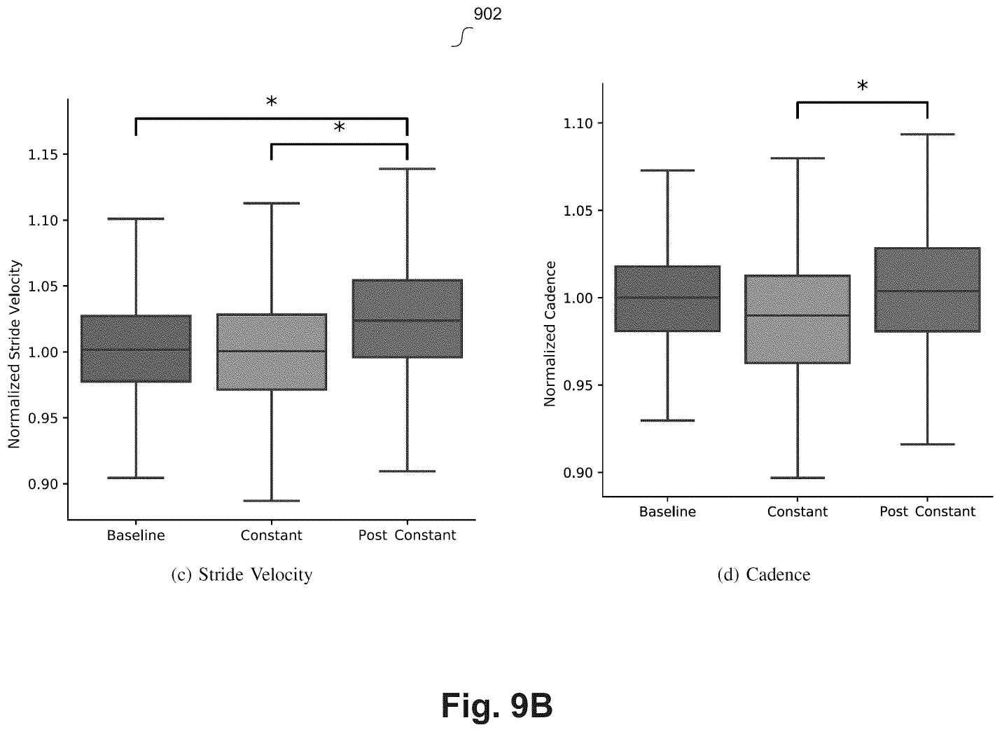

FIG. 8 and diagram 800 depict a graphical representation of gait events in a typical gait cycle. These events are used to calculate the temporal and spatial parameters of the gait. The horizontal line shows the start and end of the temporal parameters of the gait: stance time, swing time, stride time, step time. Diagram 800 also includes a graphical representation of how stride length, stride width, and step length are measured. Footprints are from the left side and the right. The dashed arrow is the walking direction. The black lines are the different measurements.

Along with the temporal and spatial parameters, two velocity parameters were used. Stride velocity (SV) is the average velocity of one side, this is SL divided by TT. Cadence (CE) is the average number of steps per minute. A step is the period from heel strike from one foot to the next heel strike of the other foot

Statistical Analysis: For each parameter, two analyses were performed. The first was to compare the changes in the sessions BA, CV, and PC to understand the effects of constant vibration on gait. To understand the effects of the gait cycle percentage timed vibration on the gait of the subjects, BA, TC, and PT sessions were compared. Data for SL, SW, TL, and SV were normally distributed, as indicated by the Kolgomorov-Smirnov test and Q-Q plots. Thus, 1-way repeated measurement ANOVA (rmANOVA) statistical analyses were used. The rmANOVA test was used to evaluate significant differences among the sessions for each of the corresponding vibration types. In the case of significance, pairwise comparison tests were carried out as post-hoc testing using Bonferroni corrections for the number of comparisons.

Data for ST, WT, TT, and CE were not normally distributed, as indicated by the Kolgomorov-Smirnov test and Q-Q plots. Thus, non-parametric statistical analyses were used. The Friedman test was used to evaluate significant differences among the sessions for each of the corresponding vibration types. In the case of significance, Wilcoxon signed-rank tests were carried out as post-hoc testing using Bonferroni correction for the number of comparisons. Statistical significance was defined for *:p<0.05, and tests were run using IBM SPSS Statistics 26.

Results—Constant Vibration: The subjects changed their gait during the BA, CV, and PC sessions. Table I shows the median and interquartile ranges for the gait parameters selected. The gait parameters for each subject were averaged per session and then grouped within subjects.

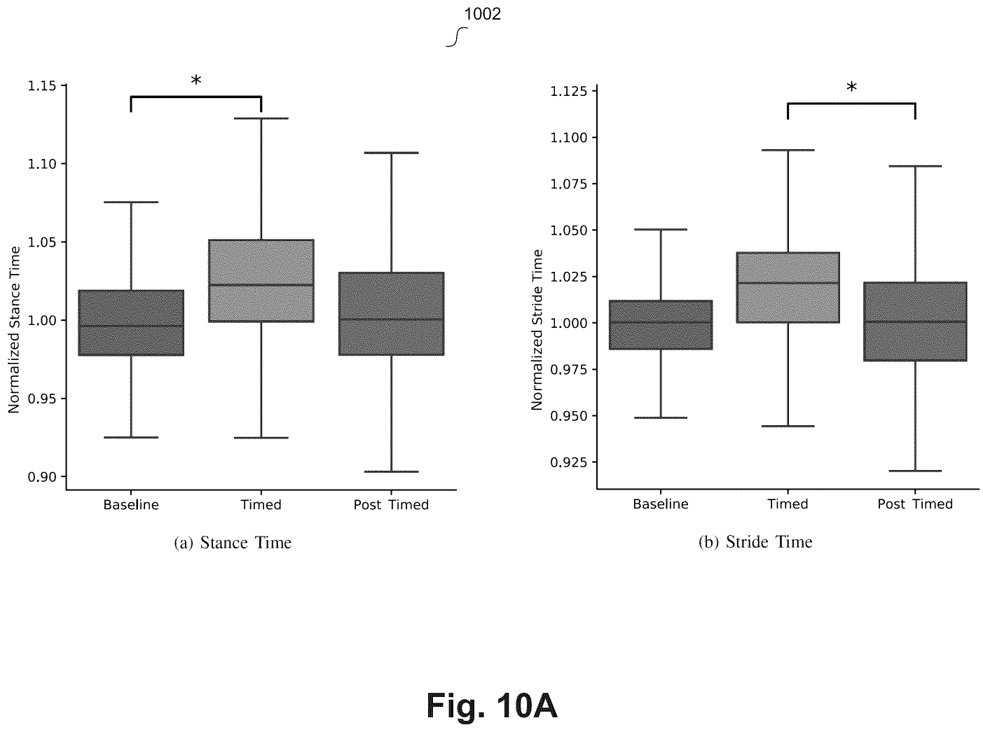

For the constant vibration analyses, main effects were found in the spatial parameters: SL(F(2,6)=7.08, p=0.023), TL (F(2,6)=7.959, p=0.006). Also, main effects were found in the velocity parameters SV (F(2,6)=6.778, p=0.011), and CE (χ(2)=6.0, p=0.049). Given that main effects were found, post-hoc pairwise comparison were carried out. For SL, BA and PC sessions were statistical different (p=0.023). For TL, statistically differences were found between the BA and PC (p=0.015) sessions. For SV, BA was different from PC (p=0.041), and CV from PC (p=0.046). Lastly, for CE, CV and PC were statistically different (z=2.673, p=0.023). Results are shown in FIGS. 9A and 9B. For SW (F(2,6), p=0.174), ST (χ(2)=2.571, p=0.276), TT (χ(2)=3.714, p=0.156), and WT (χ(2)=2.0, p=0.368) no statistical differences were found.

FIGS. 9A and 9B illustrate box plots 902 and 904 of the gait parameters for constant vibration. The boxes show quartile distribution of the variable for each model and the whiskers show the complete population. Horizontal lines are drawn between models that showed statistical significance. Values were normalized per subject, using the mean value during the baseline session.

TABLE I

| | Baseline | Constant Vibration | Post Constant |
|---|---|---|---|
| Stride Length (cm) | 135.1 ± 10.9 | 136.6 ± 12.4 | 137.7 ± 12.2 |
| Stride Width (cm) | 11.2 ± 6.7 | 10.6 ± 5.5 | 10.4 ± 5.3 |
| Step Length (cm) | 67.6 ± 5.6 | 68.3 ± 6.2 | 69.0 ± 6.1 |
| Stance Time (sec.) | 0.8 ± 0.03 | 0.8 ± 0.04 | 0.8 ± 0.04 |
| Swing Time (sec.) | 0.4 ± 0.01 | 0.4 ± 0.02 | 0.4 ± 0.02 |
| Stride Time (sec.) | 1.2 ± 0.03 | 1.2 ± 0.1 | 1.2 ± 0.04 |
| Stride | 112.7 ± 12.5 | 110.8 ± 14.8 | 114.9 ± 11.1 |
| Velocity (cm/sec.) | | | |
| Cadence (steps/sec.) | 102.9 ± 2.6 | 101.4 ± 4.0 | 104.0 ± 3.9 |

Results—Constant Vibration: The subjects changed their gait during the BA, TV, and PT sessions. Table II shows the median and interquartile ranges for the gait parameters selected. All the gait parameters for each subject were averaged per session and then grouped within subjects. For the timed vibration analyses, main effects were found in the temporal gait parameters ST (χ(2)=8.0, p=0.018), TT(χ(2)=8.0, p=0.018). Significant differences were also found in the velocity parameters SV (F(2,6)=6.310, p=0.043), and CE (χ(2)=8.0, p=0.018). Post-hoc pairwise comparison showed that for ST, BA was different from TV (z=−2.673, p 0.023). For TT, the TV session was different from PT (z=2.673, p=0.023). As for SV, TV and PT were statistically different (p=0.024). For CE, TV and PT were also statistically different (z=2.673, p 0.023). Results are shown in FIG. 7. For SL (F(2,6)=2.884, p=0.095), TL (F(2,6)=0.334, p=0.087), SW (F(2,6)=1.305, p=0.102), and WT (χ(2)=4.571, p=0.102) no statistical differences were found.

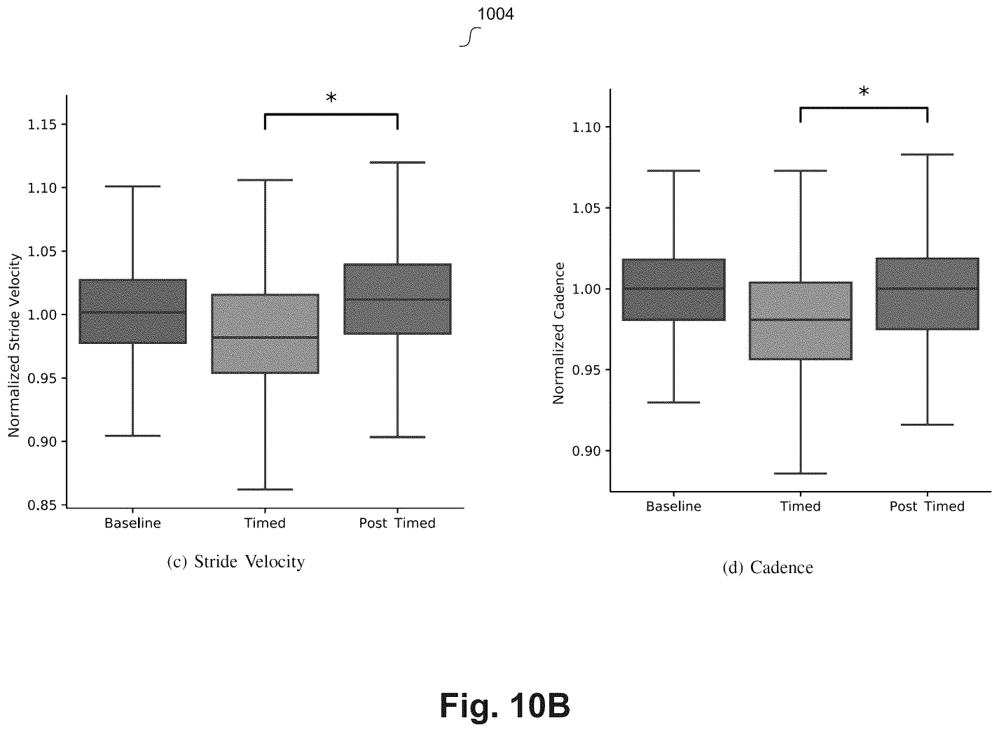

FIGS. 10A and 10B illustrate box plots 1002 and 1004 of the gait parameters for timed vibration. The boxes show quartile distribution of the variable for each model and the whiskers show the complete population. Horizontal lines are drawn between models that showed statistical significance. Values were normalized per subject, using the mean value during the baseline session.

TABLE II

| | Baseline | Timed Vibration | Post Timed |
|---|---|---|---|
| Stride Length (cm) | 135.1 ± 10.9 | 136.1 ± 12.5 | 137.3 ± 11.6 |
| Stride Width (cm) | 11.2 ± 6.7 | 10.5 ± 5.2 | 9.9 ± 5.9 |
| Step Length (cm) | 67.6 ± 5.6 | 68.1 ± 6.4 | 68.8 ± 5.9 |
| Stance Time (sec.) | 0.8 ± 0.03 | 0.8 ± 0.1 | 0.8 ± 0.1 |
| Swing Time (sec.) | 0.4 ± 0.01 | 0.4 ± 0.02 | 0.4 ± 0.02 |
| Stride Time (sec.) | 1.2 ± 0.03 | 1.2 ± 0.1 | 1.2 ± 0.1 |
| Stride Velocity (cm/sec.) | 112.7 ± 12.5 | 112.7 ± 13.0 | 116.3 ± 14.1 |
| Cadence (steps/sec.) | 102.9 ± 2.6 | 102.4 ± 4.3 | 103.9 ± 4.9 |

Discussion: The results show that the subjects modified their gait when a vibration was applied to their muscles. As for the two types of vibratory feedback, the hypothesis that the subjects would modify their cadence and stride velocity was confirmed. Both strategies found statistical differences in these two parameters. The feedback affects the gait of the subjects differently. For the constant vibration, the SV was observed to be different between PC and other sessions, and the cadence was different during the CV and PC sessions. As for the timed vibration, both the velocity and cadence were different during TV and PC.

It was observed that the constant vibration has a higher effect on the temporal parameters of the gait, making both the stride and step length statistically different. On the other hand, it was observed that the timed vibration had a greater effect on the temporal parameters of the gait, making the stance and stride times statistically different, but not the stride and step length. Both feedbacks had an observed influence on the velocity parameters.

As shown by Proske et al., the signals from the spindles change depending on the current state of the muscle and its history of activation. Therefore, the vibration feedback affects the muscle spindle differently depending on whether the muscle is being contracted or not. The timed vibration is designed to be active at the same time the muscle is active; the end result is that the vibration has a higher effect on the spatial parameters. By only providing the vibration when the muscle is being contracted, the information about the length of the muscle is affected.

Furthermore, during the constant vibration sessions, vibration is provided to the muscles at once. This means that the agonist and antagonist muscles are being excited by the feedback at the same time. As shown by Ribot-Ciscar et al., when a vibration is applied to muscle pairs, the central nervous system uses the difference of the information from the muscle spindles for positional information, i.e., the effect of the vibration is reduced. Given this property, the positional information is not affected by the vibration, therefore no statistical difference was observed on the spatial parameters.

The results show that the effects of the vibration carry over even when the vibration is not present anymore. This is shown by the statistical analysis, as PC and PT are the sessions where most of the gait parameters are different. This could be because the signals of the spindles change depending on the history of activation and the effects of the vibration during the training sessions carry over to the post sessions.

Conclusion: With the two types of haptic feedback, the subjects modified their gait. With both feedbacks, their stride velocity and cadence were modified. But this is because these parameters depend on both spatial and temporal parameters. Constant vibration modified the spatial parameters, while timed vibration modified the temporal parameters in the experiment. The results show that the strategy of when and where the vibration is applied can have an impact on gait. This can be explored further to create procedures that target specific parameters of the gait. This could be used for rehabilitation on different populations with gait impairments.

In some embodiments, the timed vibration can be synchronized to the period when the muscle has its highest activation. This can be done by creating a function that uses the predicted gait cycle. This can be modified in other embodiments, for example to test different strategies. For example, vibration could be given moments before the muscle will activate, or moments after the motion is complete. This could provide a better understanding on how the CNS processes the vibration. Furthermore, the number of targeted muscles could be increased and/or focus on antagonist muscle groups.

The implemented experiment was conducted on healthy young adults and both types of vibration had a significant effect on their gait. A similar experiment can be conducted with a broader population to investigate the effects on gait and balance. For example, one could investigate if exciting the muscle spindles has a significant impact on elderly people with balance problems.

One aspect of the invention is directed to a first apparatus for modifying a subject's gait. The first apparatus comprises a plurality of first transducers, a plurality of second transducers, and a processor. Each of the first transducers is configured for positioning proximate to a respective muscle of a right leg of the subject, and each of the second transducers is configured for positioning proximate to a respective muscle of a left leg of the subject. The processor is configured to track the subject's gait cycle and, based on the tracked gait cycle, actuate each of the plurality of first transducer and each of the plurality of second transducers at respective portions of the subject's gait cycle over multiple gait cycles.

In some embodiments of the first apparatus, the processor is configured so that each of the respective portions is synchronized to a respective time during which a respective corresponding muscle has its highest activation.

In some embodiments of the first apparatus, the plurality of first transducers includes a right tibialis anterior transducer, a right rectus femoris transducer, and a right biceps femoris transducer, and the plurality of second transducers includes a left tibialis anterior transducer, a left rectus femoris transducer, and a left biceps femoris transducer.

In some embodiments of the first apparatus, the plurality of first transducers includes a right tibialis anterior transducer, a right rectus femoris transducer, and a right biceps femoris transducer, and the plurality of second transducers includes a left tibialis anterior transducer, a left rectus femoris transducer, and a left biceps femoris transducer. In these embodiments, the processor is configured to (a) actuate the right tibialis anterior transducer during a first portion of the gait cycle, starting from right heel strike, (b) actuate the right rectus femoris transducer during a second portion of the gait cycle, (c) actuate the left biceps femoris transducer during a third portion of the gait cycle, (d) actuate the left tibialis anterior transducer during a fourth portion of the gait cycle, (c) actuate the left rectus femoris transducer during a fifth portion of the gait cycle, (f) actuate the right biceps femoris transducer during a sixth portion of the gait cycle. Optionally, in these embodiments, the first, second, third, fourth, fifth, and sixth portions of the gait cycle are all non-overlapping.

In some embodiments of the first apparatus, the plurality of first transducers includes a right tibialis anterior transducer, a right rectus femoris transducer, and a right biceps femoris transducer, and the plurality of second transducers includes a left tibialis anterior transducer, a left rectus femoris transducer, and a left biceps femoris transducer. In these embodiments, the processor is configured to (a) actuate the right tibialis anterior transducer from 0-10% of the gait cycle, starting from right heel strike, (b) actuate the right rectus femoris transducer from 10-30% of the gait cycle, (c) actuate the left biceps femoris transducer from 30-40% of the gait cycle, (d) actuate the left tibialis anterior transducer from 40-60% of the gait cycle, (e) actuate the left rectus femoris transducer from 60-75% of the gait cycle, and (f) actuate the right biceps femoris transducer from 87-100% of the gait cycle.

In some embodiments of the first apparatus, each of the first transducers and each of the second transducers comprises a vibrator configured to couple vibration into a respective leg muscle.

Some embodiments of the first apparatus further comprise at least two pressure sensors configured for positioning at the subject's shoe. In these embodiments, the processor is configured to track the subject's gait cycle based on signals received from the at least two pressure sensors. And optionally in these embodiments, the subject's gait cycle is tracked using a trained machine learning model that receives signals from the at least two pressure sensors as input.

Another aspect of the invention is directed to a first method for modifying a subject's gait. The first method comprises positioning each of a plurality of first transducers proximate to a respective muscle of a right leg of the subject, and positioning each of a plurality of second transducers proximate to a respective muscle of a left leg of the subject. The first method also comprises tracking the subject's gait cycle, and actuating each of the plurality of first transducer and each of the plurality of second transducers at respective portions of the subject's gait cycle over multiple gait cycles based on the tracked gait cycle.

In some instances of the first method, each of the respective portions is synchronized to a respective time during which a respective corresponding muscle has its highest activation.

In some instances of the first method, the plurality of first transducers includes a right tibialis anterior transducer, a right rectus femoris transducer, and a right biceps femoris transducer, and the plurality of second transducers includes a left tibialis anterior transducer, a left rectus femoris transducer, and a left biceps femoris transducer.

In some instances of the first method, the plurality of first transducers includes a right tibialis anterior transducer, a right rectus femoris transducer, and a right biceps femoris transducer, and the plurality of second transducers includes a left tibialis anterior transducer, a left rectus femoris transducer, and a left biceps femoris transducer. And the actuating comprises (a) actuating the right tibialis anterior transducer during a first portion of the gait cycle, starting from right heel strike, (b) actuating the right rectus femoris transducer during a second portion of the gait cycle, (c) actuating the left biceps femoris transducer during a third portion of the gait cycle, (d) actuating the left tibialis anterior transducer during a fourth portion of the gait cycle, (c) actuating the left rectus femoris transducer during a fifth portion of the gait cycle, (f) actuating the right biceps femoris transducer during a sixth portion of the gait cycle. Optionally, in these instances, the first, second, third, fourth, fifth, and sixth portions of the gait cycle are all non-overlapping.

In some instances of the first method, the plurality of first transducers includes a right tibialis anterior transducer, a right rectus femoris transducer, and a right biceps femoris transducer, and the plurality of second transducers includes a left tibialis anterior transducer, a left rectus femoris transducer, and a left biceps femoris transducer. And the actuating comprises (a) actuating the right tibialis anterior transducer from 0-10% of the gait cycle, starting from right heel strike, (b) actuating the right rectus femoris transducer from 10-30% of the gait cycle, (c) actuating the left biceps femoris transducer from 30-40% of the gait cycle, (d) actuating the left tibialis anterior transducer from 40-60% of the gait cycle, (e) actuating the left rectus femoris transducer from 60-75% of the gait cycle, and (f) actuating the right biceps femoris transducer from 87-100% of the gait cycle.

In some instances of the first method, each of the first transducers and each of the second transducers comprises a vibrator configured to couple vibration into a respective leg muscle.

In some instances of the first method, the tracking of the subject's gait cycle is based on signals received from the at least two pressure sensors. Optionally, in these instances, the subject's gait cycle is tracked using a trained machine learning model that receives signals from the at least two pressure sensors as input.

Another aspect of the invention is direct to a second apparatus for modifying a subject's gait. The second apparatus comprises at least two pressure sensors and a first transducer configured for positioning proximate to a first leg muscle of the subject. The second apparatus also comprises a processor configured to track the subject's gait cycle responsively to signals from the at least two pressure sensors and, based on the tracked gait cycle, actuate the first transducer to provide feedback to the subject in synchronicity with a first portion of the subject's gait cycle.

In some embodiments of the second apparatus, the processor is configured to actuate the first transducer in synchronicity with the first portion of the subject's gait cycle over multiple gait cycles. In some embodiments of the second apparatus, the first transducer comprises a vibrator configured to couple vibration into the first leg muscle.

In some embodiments of the second apparatus, the subject's gait cycle is tracked using a trained machine learning model that receives signals from the at least two pressure sensors as input.

Some embodiments of the second apparatus further comprise a second transducer configured for positioning proximate to a second leg muscle of the subject, and the processor is configured to actuate the second transducer to provide feedback to the subject in synchronicity with a second portion of the subject's gait cycle.

In some embodiments of the second apparatus, the processor is configured to actuate the first transducer in synchronicity with the first portion of the subject's gait cycle and the second transducer in synchronicity with the second portion of the subject's gait cycle over multiple gait cycles. In some embodiments of the second apparatus, the first portion of the subject's gait cycle does not overlap with the second portion of the subject's gait cycle.

In some embodiments of the second apparatus, the feedback comprises haptic feedback at a frequency between 60 and 100 Hz. In some embodiments of the second apparatus, the at least two pressure sensors are configured for positioning at the subject's shoe. In some embodiments of the second apparatus, the apparatus comprises an accelerometer, and the processor is configured to track the subject's gait cycle responsively to signals from the at least two pressure sensors and the accelerometer.

In some embodiments of the second apparatus, the processor is configured to actuate the first transducer upon detection of a change in stride characteristic of the tracked gait cycle. In some embodiments of the second apparatus, the processor is configured to actuate the first transducer upon detection of a change in stride length of the tracked gait cycle.

Another aspect of the invention is direct to a second method for modifying a subject's gait. The second method comprises positioning a first transducer proximate to a first leg muscle of a subject, tracking the subject's gait cycle, and based on the tracked gait cycle, providing feedback to the first leg muscle by actuating the first transducer in synchronicity with a first portion of the subject's gait cycle.

In some instances of the second method, the actuating of the first transducer in synchronicity with the first portion of the subject's gait cycle causes a modification to the subject's gait. In some instances of the second method, the first transducer is actuated in synchronicity with the first portion of the subject's gait cycle over multiple gait cycles.

In some instances of the second method, the subject's gait cycle is tracked using a trained machine learning model. In some instances of the second method, the trained machine learning model is configured to track the subject's gait using signals received from a set of sensors over multiple gait cycles, and the set of sensors comprises one of a) two pressure sensors, or b) a pressure sensor and an accelerometer.

Some instances of the second method further comprise positioning a second transducer proximate to a second leg muscle of a subject, and, based on the tracked gait cycle, providing feedback to the second leg muscle by actuating the second transducer in synchronicity with a second portion of the subject's gait cycle.

In some instances of the second method, the first portion of the subject's gait cycle does not overlap with the second portion of the subject's gait cycle. In some instances of the second method, at least some of the first portion of the subject's gait cycle overlaps with the second portion of the subject's gait cycle. In some instances of the second method, the first transducer is actuated in synchronicity with the first portion of the subject's gait cycle and the second transducer is actuated in synchronicity with the second portion of the subject's gait cycle over multiple gait cycles.

In some instances of the second method, the first transducer is actuated upon detection of a change in stride characteristic of the tracked gait cycle. In some instances of the second method, the first transducer is actuated upon detection of a change in stride length of the tracked gait cycle.

### Section II

Freezing of gait is an episodic phenomena faced by many patients with Parkinson's disease. It is characterized by episodes during which patients are unable to generate effective forward stepping movements, despite absence of motor deficits. During the onset of the event, the patients are less stable with statistically different stride width, toe in/out angle and center of pressure distance. It has been postulated that the degree of freezing can be reduced by providing external sensory feedback to the patients during the event. This intervention could be facilitated by accurate identification of freezing events in real-time.

Embodiments present an Artificial Neural Network model which uses signals recorded by an instrumented footwear to predict if a walking subject is having a freezing episode. Embodiments of the model are capable of continuously predicting freezing of gait events at a high temporal resolution of 50 Hz, using a 0.5 second window of data recorded by the instrumented shoes, with a sensitivity of 96.0±2.5%, a specificity of 99.6±0.3%, a precision of 89.5±5.9%, and an accuracy of 99.5±0.4%. This algorithm was tested with data collected from 10 patients with Parkinson's disease with frequent freezing of gait episodes.

Parkinson's disease (PD) is the second most common neurodegenerative disorder in the world, affecting about 1% of the population over 60 years of age. Depending on the stage of the disease, between 20-60% of individuals with PD suffer from episodic freezing of gait (FOG). FOG is a motor phenomenon where ambulation is attempted, yet halts for several seconds in a transient period. These transient periods of halted gait have been specifically described as “brief episodes during which patients find it impossible to generate effective forward stepping movements, in the absence of a cause other than parkinsonism or higher cortical deficits.” When an individual experiences FOG, they will remain in place, even while attempting to ambulate forward or complete steps. This is analagous to the individual's feet being “glued” to the floor. When the individual overcomes the FOG, they return to typical ambulation at their normal pace until the next freezing episode develops.

Although FOG limits the natural movement of the legs during gait, FOG is not related to muscle weakness. FOG is actually most commonly experienced when the individual is turning, initiating a new step, or is faced with an external constraint such as a doorway, distraction, or stress. Providing external stimuli, or cues, and focusing attention on forward ambulation can aid individuals in overcoming episodes of FOG. However, the unpredictable nature of FOG episodes often leads to an increase in falls. Therefore, not only does FOG reduce mobility for some PD patients, it also has other clinical implications, like loss of independence, recurrent falls, and related physical injuries.

The duration of FOG events is typically less than 10 seconds. However, as the disease progresses, individuals encounter more frequent FOG episodes and have increased difficulty overcoming the episodes. During an episode, a loss of balance can be experienced, leading to an increased risk of falling. The increased frequency of falls among individuals with PD are intrinsic to the disease, and may not be linked to their environment. Although not necessarily linked to environment, FOG episodes are unpredictable. The influence of attention, external cues, and sensory stimuli make it difficult to reproduce FOG episodes in clinical or research settings. Studies have shown that the gait of PD patients who present FOG is different from those patients who do not present the events. Hausdorff et al. showed that PD patients that present FOG have a higher stride-to-stride variability on the stride time.

Furthermore, Nieuwboer et al. showed that even for the same patients, the gait is different during the onset of the event. In the study, the patients walked in a clinical setting. During the onset of the FOG, the cadence was significantly different and the stride length decreased. Dopaminergic medications are typically prescribed to treat most motor symptoms associated with PD, including rigidity, bradykinesia and tremors. However, FOG episodes are notoriously resistant to levodopa, a traditional dopaminergic medication, especially as the severity of PD progresses. Other forms of non-pharmacological interventions have potential in preventing or reducing FOG episodes. Recent work have reported reductions in the frequency of FOG events when auditory, visual, and haptic feedback are provided to patients.

Wearable devices can be used to study the effects of these various feedback methods on FOG events, even in novel environments outside of a clinical setting. These devices vary in instrumentation and location; some contain at least one inertial measurement unit (IMU) mounted to different anatomical locations such as the pelvis, back, and upper and lower extremities. The portability and instrumentation of wearable devices can allow quantitative and continuous monitoring of individuals with PD during daily tasks. Not only can these devices passively monitor individuals, but the detection of FOG events by these devices would allow for external sensory feedback to be provided during an episode.

Related Work: To move towards identification of FOG events from passive collection of sensor data, wearable devices can use algorithms which process the sensor readings. Characteristic features from sensor signals, which could be spatial, temporal, or in the frequency domain, can be analyzed and identified. Once these features are identified, rules can be set to detect FOG events. This process of identifying and detecting signal features from sensor data involved detailed processing and feature extraction from each sensor. The increase of sensors may potentially improve the reliability in event detection. However, the complexity of the associated algorithms increases with each additional sensor. A summary of the state-of-the-art for the identification of FOG events using wearable devices is shown in Table III.

TABLE III

| | Data | | | | | | | |
|---|---|---|---|---|---|---|---|---|
| | set | Sensor | | | | | | |
| Author | Size | Location | Sensor Type | Algorithm | Sensitivity | Specificity | Precision | Accuracy |
| Capecci | 20 | Waist | Accelerometer | Thresholding | 87.5% | 94.9% | 69.5% | 84.3% |
| Rezvanian | 10 | Shin | Accelerometer | Continuous | 84.9% | 81.0% | N/R | N/R |
| | | | | Wavelet | | | | |
| | | | | Transform | | | | |
| Ahlrichs | 20 | Waist | Accelerometer | Support Vector | 92.3% | 100% | N/R | N/R |
| | | | | Machine | | | | |
| Martin | 6 | Waist | Accelerometer | Support Vector | 91.7% | 87.4% | N/R | N/R |
| | | | | Machine | | | | |
| Tripolity | 16 | 8 sensors, | Accelerometer, | Random Forests | 81.9% | 98.7% | 96.1% | 89.0% |
| | | full body | Gyro-scope | | | | | |
| Mazilu | 12 | Chest, | Electrocardiography, | Thresholding | 76.2% | N/R | 66.0% | N/R |
| | | fingers | Skin-conductance | | | | | |
| El-Attar | 10 | Shank, | IMU | ANN | 100% | 87.5% | 88.9% | 93.8% |
| | | thigh, hip | | | | | | |
| Marcante | 10 | Feet | Pressure and | Rule based | 96.0% | 94.0% | N/R | N/R |
| | | | Accelerometer | | | | | |
| Xia | 10 | Shank, | Accelerometer | ANN | 69.3% | 90.6% | N/R | 80.7% |
| | | thigh, hip | | | | | | |

Capecci et al. developed an algorithm which detects FOG events using onboard smartphone sensors for twenty individuals with PD. The phone was secured to the waist with an elastic belt. The phone's measured acceleration was used to calculate the Fast Fourier Transform (FFT) within a window of 2.5 seconds and identify its power spectrum. To minimize the complexity, only the vertical accelerations were considered and thresholding of the variables was implemented. The threshold value was tuned per subject to detect the maximum number of FOG events.

Ahlrichs et al. and Martin et al. also used the accelerations at the pelvis to detect FOG, but instead implemented a Support Vector Machine algorithm. Both algorithms utilized the FFT of the same dataset and identified the optimal time window using a variable window size. Ahlrichs also implemented a combination of thresholds to the features to improve the classification performance. To use the acceleration data for classification, the signals were resampled, filtered, and the FFT was calculated. This preprocessing negatively affects the prediction speed.

Rezvanian et al. placed their accelerometer at the shin and used a Continuous Wavelet Transform (CWT) to identify the FOG episodes. For their CWT, a 2 second window was used, and a threshold per subject was defined. The acceleration data were filtered and resampled, and their prediction was capped at 2 Hz, or 0.5 seconds. Tripolity et al. used six accelerometers and two gyroscopes. They compared four machine learning algorithms and used the entropy as the input feature. Their algorithms used a window of 1 second with 0.5 seconds of overlap. Their results showed that Random Forests gave the best performance for their collected dataset.

The prior models rely on extensive data preprocessing for an optimal identification performance. The required data preprocessing impacts the speed of event identification, limiting the ability of the algorithm to deliver real-time feedback. The ability to detect and identify FOG events in less time would allow for real-time feedback of external sensory cues.

Artificial Neural Networks (ANN) are models which can map an input to a class. Supervised learning is used in ANNs, where each training input has a true known output. This allows for the calculation of weights which map the input to the output. To determine this mapping, a cost function is minimized through stochastic gradient descent and backpropagation. These allow the network to analyze multiple signals in a single step, without increasing the network complexity and minimal impact to the computational load. Not only can they analyze multiple signals, but spatial and temporal data can also be combined. This training is computationally expensive, but the compiled mapping of the signals can be done rapidly, without any preprocessing.

Lorenzi et al. identified common behaviors of FOG using an ANN. These behaviors included short steps, stopping during gait, and trunk fluctuations. The authors implemented a shallow network of just two layers to minimize the computation time, and used raw accelerometer data with only fully connected layers. This algorithm was implemented on healthy individuals; the behavior identification still needs to be evaluated on individuals with PD.

El-Attar et al. detected FOG events by combining a Discrete Wavelet Transform (DWT) and ANNs. They also used a shallow network with two layers and 20 neurons. Their algorithm was able to achieve a sensitivity of 100% using data from ten patients with PD, but their dataset was augmented with frequency domain information identified separately from the dataset. When selecting the parameters of an ANN, different types of layers can be used to encode information in order to avoid data preprocessing. Convolutional Neural Networks (CNN) use the convolution operator to find the kernel parameters automatically, reducing noise by encoding and decoding the data. This method can successfully use IMU signals to identify human motion.

Recurrent Neural Networks (RNN) capture time dependencies in the data and generate sequence-to-sequence mapping. RNN models use leaky units to help the network maintain its state, accumulate data over time, and forget the previous states when they are no longer relevant. Ashour et al. showed that recurrent neural networks outperform traditional machine learning methods at identifying FOG events. Furthermore, they showed that the raw data from IMU can be used without any feature extraction.

Embodiments include an ANN model that can predict if a FOG event will occur. A proposed model combines signals recorded by embodiments of a custom instrumented footwear. Embodiments of the model maintain a high temporal resolution while continuously predicting FOG. The results of using this model to predict FOG episodes are presented using data from 10 patients with PD.

Dataset Description—Experiment Design: In an example implementation, the training dataset contained data from 10 subjects with Parkinson's Disease (6 males and 4 females, with 10.5±6.63 years of PD and a Hoehn and Yahr stage of 2.8±0.7) who exhibited FOG episodes during the recording. The participant characteristics are shown in Table IV.

TABLE IV

| | | Age | Weight | Height | Reported | Years | H&Y |
|---|---|---|---|---|---|---|---|
| SubID | Gender | (yrs) | (kg) | (m) | FOG | of PD | stage |
| Sub001 | Male | 57 | 72 | 1.67 | Yes | 11 | 3 |
| Sub002 | Male | 69 | 86 | 1.8 | Yes | 2 | 4 |
| Sub003 | Male | 78 | 76 | 1.7 | Yes | 4 | 4 |
| Sub004 | Male | 67 | 82 | 1.8 | No | 8 | 3 |
| Sub005 | Male | 63 | 84 | 1.75 | Yes | 14 | 2 |
| Sub006 | Female | 71 | 69 | 1.55 | Yes | 14 | 2 |
| Sub007 | Female | 55 | 68 | 1.7 | Yes | 14 | 2 |
| Sub008 | Male | 76 | 93 | 1.78 | No | 5 | 2 |
| Sub009 | Female | 68 | 68 | 1.62 | Yes | 7 | 3 |
| Sub010 | Female | 75 | 53 | 1.55 | Yes | 26 | 3 |

Each participant walked in multiple continuous laps on a 7 meter Zeno Walkway (Protokinetics, PA, USA) during 6 minutes. Once the participant reached the end of the walkway, they would turn around and walk back across the mat to where they began. This procedure was repeated for the duration of the recording. If the participant felt tired, they were allowed to rest between laps until they recovered. One video camera was placed at each end of the walkway and an investigator followed the subject as they walked with a third video camera focused on the participant's feet.

The subjects wore an embodiment of the DeepSole System over the duration of the experiment. In some embodiments, the DeepSole system, shown in FIG. 3, is a soft, flexible, instrumented insole which can be comfortably inserted into standard footwear. Some embodiments collect signals from twelve channels: three pressure signals, three linear accelerations, three angular velocities, and three Euler angles. The pressure sensors can be made with piezo resistive textiles (e.g., cut to the shape of the insole). The sensors can be located under the phalanges, the metatarsals, and the calcaneus. The resistance of the fabric decreases when the applied force increases on the sensing area. By placing the sensors in the aforementioned locations, loading changes during the gait can be captured. The accelerations, angular velocity, and Euler angles can be measured in the local IMU coordinate system. The sensor readings can be recorded (e.g., at 50 Hz on an on-board microSD card) and streamed (e.g., through WiFi using User Datagram Protocol (UDP) data packets).

In the example implementation, the video cameras and the DeepSole System were synced to the Zeno Walkway time by using a custom circuit that turns on a light and broadcasts a UDP packet through the network at the start of the session. The light was located in a place visible to all three video cameras and the DeepSole system recorded the time when the sync UDP was received. The Zeno Walkway was used to synchronize the recording and standardize the length of the distance traveled per lap for each subject.

Dataset Description—Gait Parameters: Ten gait parameters were selected to analyze the differences between the laps where the subjects presented a FOG and where the FOG was not present. These parameters were obtained from the Zeno Walkway software (PKMAS) in the example implementation. The strides used in this analysis were the strides performed on the instrumented mat. Stride length (SL), stride width (SW), and step length (SpL) were chosen to see the effects on the spatial characteristic of the gait. For the temporal characteristics, stance time (SaT), swing time (SwT), step time (SpT), and stride time (ST) were chosen. For balance parameters, toe in/out angle (TA) and center of pressure distance (CD) during stance were chosen. Temporal parameters and spatial parameters are shown in FIG. 8. The mean and standard deviation of these gait parameters are shown in Table V.

TABLE V

| | REG | FOG |
|---|---|---|
| Step Length (cm.) | 54.0 ± 12.5 | 47.3 ± 10.5 |
| Stride Length (cm.) | 107.4 ± 23.9 | 94.5 ± 19.6 |
| Stride Width (cm.) | 8.3 ± 4.4 | 8.0 ± 4.4 |
| Stance Time (sec.) | 0.8 ± 0.2 | 0.8 ± 0.3 |
| Swing Time (sec.) | 0.4 ± 0.2 | 0.4 ± 0.1 |
| Toe In/Out Angle (deg.) | 5.0 ± 23.0 | 9.7 ± 8.7 |
| COP Distance (cm.) | 27.1 ± 5.5 | 26.0 ± 4.7 |
| Step Time (sec.) | 0.6 ± 0.2 | 0.6 ± 0.2 |
| Stride Time (sec.) | 1.1 ± 0.3 | 1.2 ± 0.3 |

The progression vector can be from the location of the heel strike from one side to the subsequent heel strike of the same side. The norm of this vector is the SL. SW is defined as the perpendicular distance from the progression vector to the heel strike of the opposite side. SpL is defined as the distance from the location of the heel strike to the heel strike of the opposite side, measured along the progression vector.

The TA is the angle between the direction of progression and the line connecting the heel and the toe of the foot. This parameter has an impact on the base of support and can affect the balance of the person. CD is the distance the center of pressure moves during the stance phase. This is a measure of how stable the person was while moving forward. SaT is the duration that the corresponding foot is in contact with the ground, this is between heel strike and toe off. SwT is the duration between toe off to the subsequent heel strike of the same side, i.e., the period that the foot is not in contact with the floor. SpT is the time from heel strike until the heel strike of the other side. ST is the time from heel strike to heel strike of the same side, this also corresponds to SaT+SwT. Other suitable gait parameters can be implemented in other embodiments.

Dataset Description—Gait Parameters Statistical Analysis: The subjects walked a total of 323 laps on the walkway. From those, the subjects presented a FOG event while turning in 108 laps, and did not present a FOG event in 215 laps. A statistical study was done on the gait parameters to test if the gait of the subject was different a few strides before their motion was impaired. For SL, SW, SwT, SpL, TA, and CD data were normally distributed, as indicated by the Kolgomorov-Smirnov test and Q-Q plots. Thus, a repeated measurements ANOVA test was used to evaluate significant differences among the FOG laps and the non FOG laps. For SaT, ST, and SpT data were not normally distributed, as indicated by the Kolgomorov-Smirnov test and Q-Q plots. Thus, non-parametric statistical analyses were used. The Kruskal-Wallis test was used to evaluate significant differences among the FOG laps and the non FOG laps. Statistical significance was defined for *:p<0.05, and tests were run using stats models Python module. For the normally distributed parameters, the F ratio and significance level (p-value) are presented for each variable. For the nonparametric test, the test statistic (χ2) and significance level are shown.

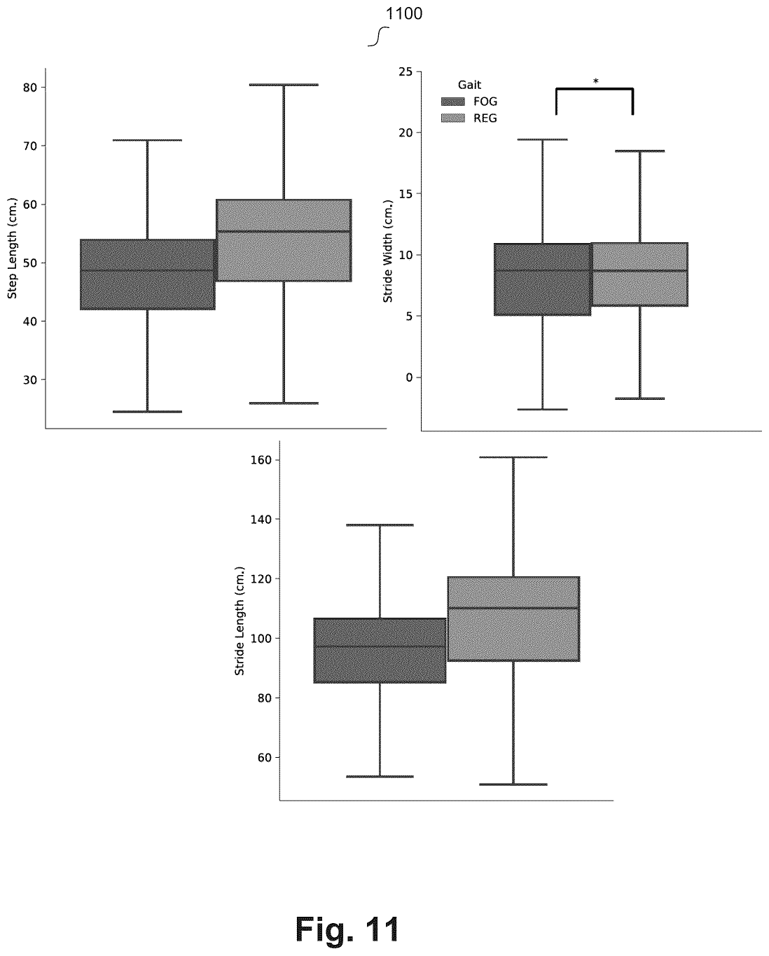

For the spatial parameters, SpL (F=0.63, p=0.45) and SL (F=0.71, p=0.42) were not statistically different. But SW (F=8.50, p=0.02) was statistically smaller before the subject was affected by a FOG event. FIG. 11 illustrates box plots 1100 for the spatial parameters of ten PD patients. Left boxes are the median and interquartile range of the laps where a FOG event was present, and right boxes represent the laps where no FOG events were registered. Statistical significance is show with *:p<0:05. Step length, stride width and stride length were all normally distributed.

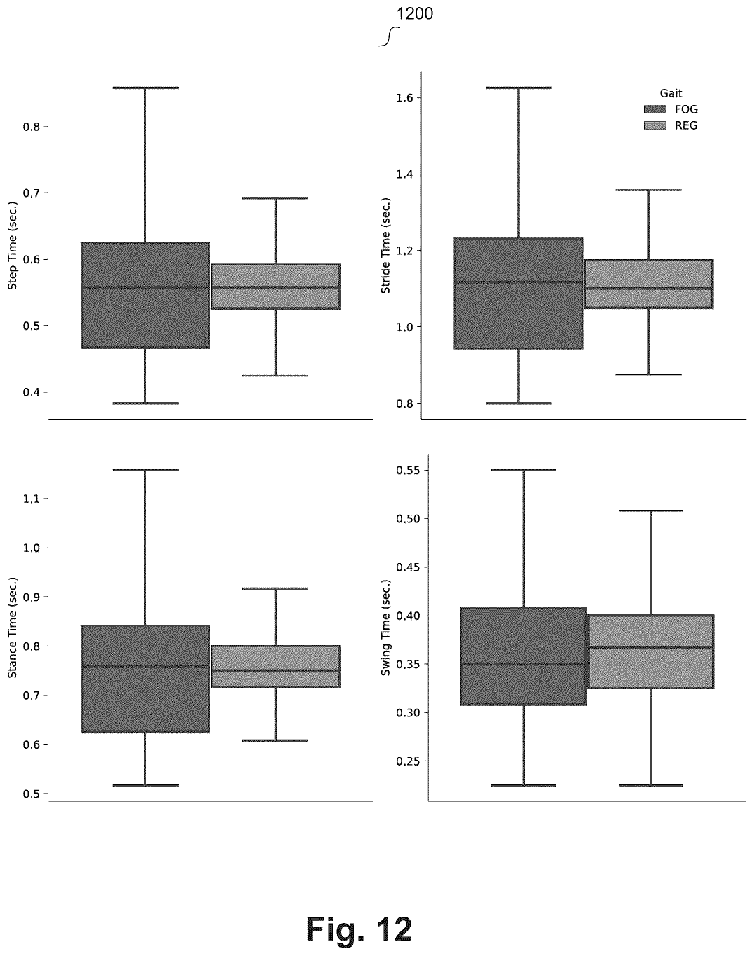

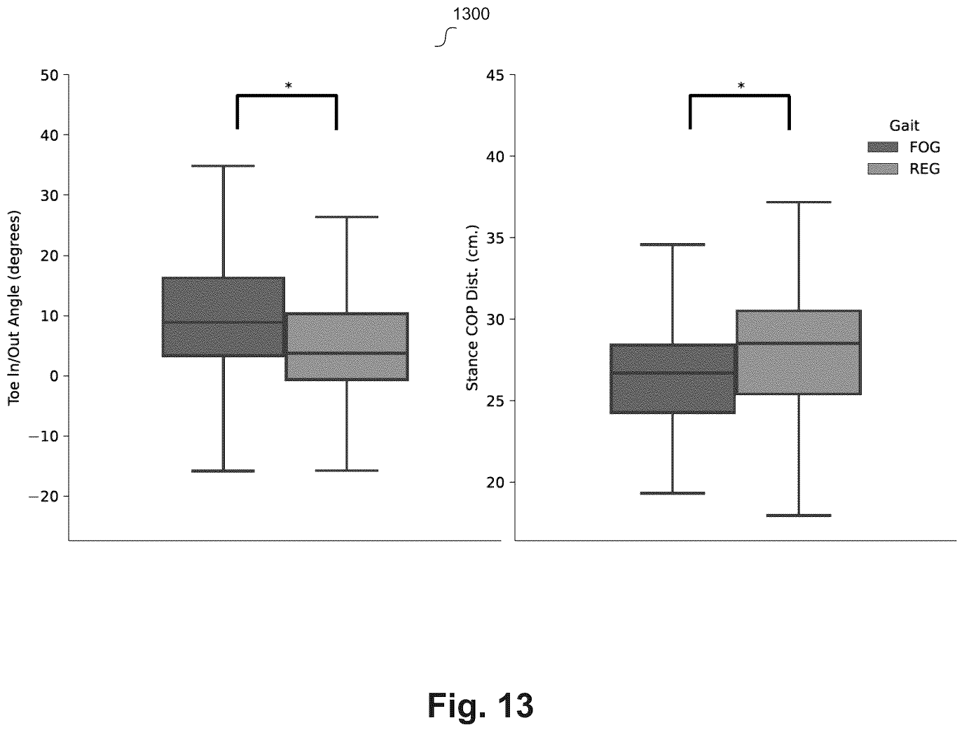

For the balance parameters, TA (F=8.68, p=0.01) was statistically larger and CD (F=9.45, p=0.01) was statistically smaller. FIG. 12 illustrates box plots 1200 for the balance parameters of ten PD patients. Left boxes are the median and interquartile range of the laps where a FOG event was present, and right boxes represent the laps where no FOG events were registered. Statistical significance is show with *:p<0:05. As for the temporal parameters, neither SaT (χ2(1)=0.001, p=0.97), SwT (F=3.65, p=0.09), SpT (χ2(1)=0.01, p=0.92), nor ST (χ2(1)=0.001, p=0.97) were significantly different. FIG. 13 illustrates box plots 1300 for the temporal parameters of ten PD patients. Left boxes are the median and interquartile range of the laps where a FOG event was present, and right boxes represent the laps where no FOG events were registered. Statistical significance is show with *:p<0:05. Swing time was normally distributed. Stance time, step time, and stride time were nonnormally distributed.

The statistical study shows that the gait of people who present FOG is different before they present the event. This is true even within small number of steps per lap (8.3±3.44 average steps per lap). This result allows us to do a more granular type of identification, where we can use a DeepSole sensors to predict the current state of the patient, i.e., if the patient is currently in a FOG state.

Dataset Description—Sensor Segmentation: In light of the results from the statistical analysis performed in the previous section, it is confirmed that the balance parameters and SW of the patient's gait is different in the laps where a FOG event occurred from those where the episode did not occur. This property can be used to map the signal from the DeepSole sensors to the current state of the gait (freezing or regular gait). To achieve this, the data can be segmented into windows.

For each time t, embodiments of the Deepsole System can record sensor values (e.g., twelve sensor values) for both the left and right foot. To identify the FOG, a window of 0.5 seconds was used to predict if the wearer will have a FOG event at time t+dt. The 0.5 second window was chosen because it represents approximately half a cycle. This amount has been found to be enough to represent the time history of a gait event. Furthermore, by using this window size, embodiments of the neural network can be computed at the same rate as embodiments of the DeepSole sampling rate.

Given that some embodiments of DeepSole record data at 50 Hz, at each time t, two matrices of size R25×12 were created. Each column represents a sensor signal and the row number corresponds to the time. To use with embodiments of the neural network, the matrices from the right and left shoes were stacked into a 3D tensor of shape R25×2×12. The last row is the latest reading from the sensors and the first is the reading at t−49dt.

Dataset Description—Identifying the Freezing of Gait Events: The video recordings were used to identify the FOG events. A clinical expert coded the videos to identify when the FOG episode started and when it stopped. The video was coded with a resolution of 1 second. This code was transformed into a continuous binary signal supersampled to the DeepSole System time, Eq. (1). In this signal, a value of 0 represents regular gait (REG) and a value of 1 represents FOG.

$${y}_{T} ( t ) = \{ \begin{matrix}0 & ( REG ) \\ 1 & ( FoG )\end{matrix} \qquad ( 1 )$$

In the example implementation, Eq. (1) was used as the ground truth for the supervised learning of the ANN by pairing each value of yT(t) with the corresponding sensor reading from an embodiment of DeepSole. The video coding includes the continuous walking and the turning at the end of the walkway.

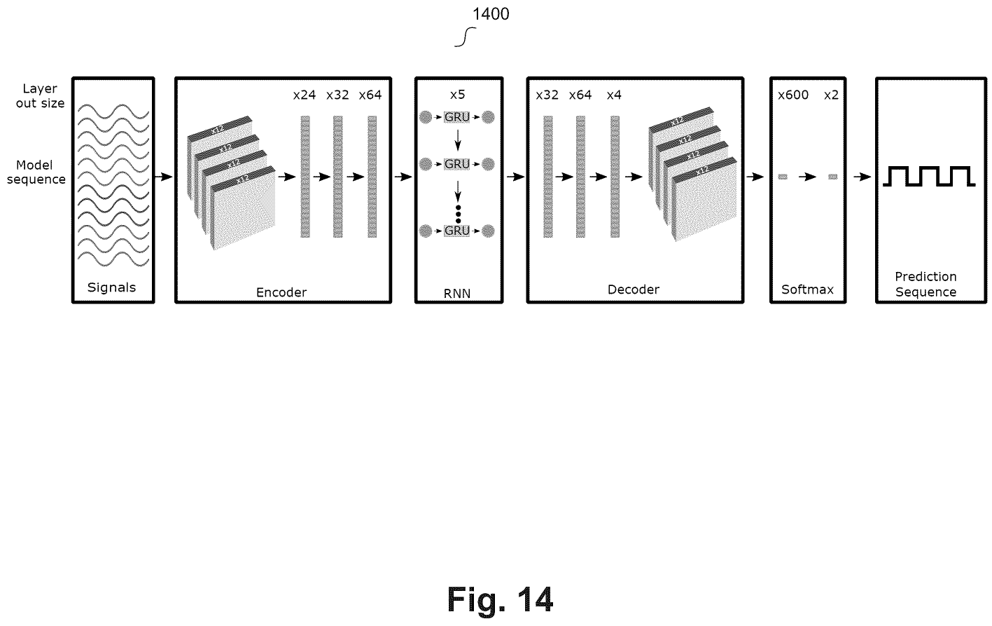

Neural Network Design: To predict if the wearer has a FOG episode given the sensor signals recorded from the embodiment of the DeepSole System, an ANN was created. A combination of a 2D CNN to encode-decode the signals and RNN to learn the temporal relation in the sensor readings was used in the example implementation. FIG. 14 illustrates and overview of neural network 1400 with an Encoder-Decoder RNN architecture. In some embodiments, model maps 12 signals collected by embodiments of the DeepSole system into the predicted FOG episode. A value of 0 corresponds to regular gait and a value of 1 corresponds to freezing. To train embodiments of the network, the aforementioned dataset was used as supervised learning, where each input has a corresponding truth output. During training, the initial 75% of the data recorded was used for training and the last 25% was used for evaluation. An embodiment of the model was implemented using Tensorflow.

In an example implementation, the model starts with an encoder with four 2D CNN layers. Dimensions of the inputs can be kept constant throughout the convolution layers by setting the number of filters to 12 and the paddings to the same number. The rectified linear unit (RcLU) function can be used at each layer. The kernel size can be 30 for layer one, 20 for layer two, 10 for layer three, and 5 for layer four. After the convolution, the outputs from the CNN can be reshaped from R25×2×12 to R25×24 and passed through two fully-connected layers with 32 and 64 units respectively, with a ReLu activation.

The outputs from the fully-connected layers can be fed into a recurrent layer. The recurrent layer contains 5 Gated Recurrent Unit (GRU) cells with a ReLu activation. After the recurrent layers, three fully-connected layers can be used with 32, 64, and 4 neurons respectively. The outputs can then be reshaped back into R25×2×2 and passed through a 2D CNN decoder of four layers. Again, the dimensions can be kept constant within the convolutions and a kernel size of 30, 20, 10, and 5 can be used respectively for the layers.

Finally, the output can be flattened and passed to a fully connected layer with 2 neurons and a softmax activation to create a probability vector of FOG. The class with the highest probability can be chosen as the final output yP(t). In the example implementation, the model was trained for 200 epochs. For each window presented, the corresponding value of yT(t) was presented. The loss used can be sparse categorical crossentropy. The loss can be minimized by applying the Adam optimizer. A learning rate of 1e-4 can be used and 50% dropout can be used throughout the models to avoid over-fitting. Other embodiments of the model can use different architectures, layers, layer sizes, data structure sizes, activation functions, algorithms, training data, input data, loss functions, optimizations, and other suitable machine learning characteristics.

Metrics: The binary FOG function yT(t) (ground truth) was compared against the output from the ANN yP(t) (predicted event) to evaluate the performance of embodiments of the ANN. The number of correctly identified episodes of FOG was labeled as True Positives (TP) and the number of incorrectly FOG identified was labeled as False Positives (FP). Similarly for REG events, the correctly identified events were labeled as True Negatives (TN) and the misidentified were labeled as False Negatives (FN). The FP and FN percentages were calculated using the ratio of the false events divided by the corresponding total of events identified by the ground truth, these percentages were not used on the metrics calculations.

Four parameters were evaluated to assess the performance of the networks:
1) Sensitivity: The proportion of FOG samples correctly identified in yP divided by the total FOG samples in yT.

$$Sensitivity = \frac{T P}{T P + F N} \times 100 \qquad ( 2 )$$

2) Specificity: The number of REG samples correctly identified in yP divided by the total REG samples in yT.

$$Specificity = \frac{T N}{T N + F P} \times 100 \qquad ( 3 )$$

3) Precision: The number of FOG samples correctly identified in yP with respect to the total number FOG samples identified by yP.

$$Precision = \frac{T P}{T P + F P} \times 100 \qquad ( 4 )$$

4) Accuracy: The percentage accuracy between the ground truth and the predicted output was calculated as the proportion of correct prediction to the total number of samples.

$$Accuracy = \frac{T P + T N}{T P + F N + T N + F P} \times 100 \qquad ( 5 )$$

For the calculations, yT and yP were used at the original 50 Hz that an embodiment of DeepSole recorded the data. This was done to identify the FOG episodes as soon as possible in real-time.

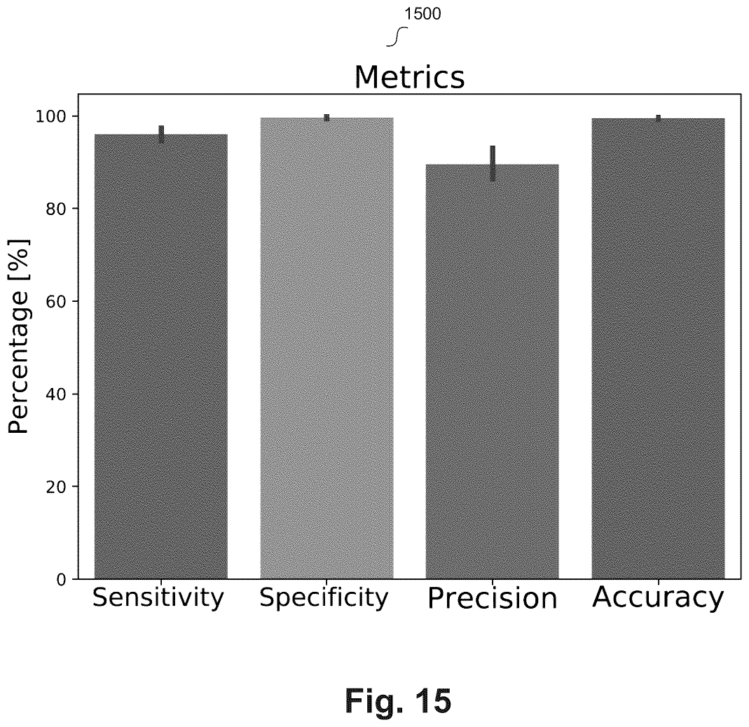

Results: Data was aggregated and prediction metrics were calculated. The data used was the last 25% of the data recorded for each subject, as this data was not used during training. Table VI contains a summary of the results. FIG. 15 shows bar plots 1500 of the four metrics used to evaluate the model. The height is the average among subjects and the whiskers are plus or minus one standard deviation of the interpatient variation. For both the table and the figure, the values is the interpatient variation. The model sensitivity was 96.0±2.5% FOG events, but 11.6±6.9% of the FOG events were mislabeled (False Positives). The precision was 89.5±5.9%. This represents the ability of the model to identify the FOG samples. An embodiment of the model was able to identify the FOG episodes that occurred during straight walking and during turning with high sensitivity and precision. This was true for all subjects in the dataset. The presented model outperforms state-of-the-art methods for precision. For sensitivity, only El-Attar has a higher rate, but they use a higher number of sensors.

TABLE VI

| Metric | Mean ± SD |
|---|---|
| Sensitivity | 96.0 ± 2.5% |
| Specificity | 99.6 ± 0.3% |
| Precision | 89.5 ± 5.9% |
| Accuracy | 99.5 ± 0.4% |
| False Positives | 11.6 ± 6.9% |
| False Negatives | 0.2 ± 0.2% |

For the REG events, the model specificity was 99.6±0.3%, but 0.2±0.2% were incorrectly identified (False Negatives). This shows that an embodiment of the model excels at not identifying regular gait as FOG. This is especially useful at the edges of the walkway, when the subject is turning. During turning, the subject stops and turns, but the model correctly identifies this REG without information about the frequency domain. This metric is also better than the current state-of-the-art, almost matching the 100% specificity reported by Ahlrichs et al. The overall accuracy of the prediction was 99.5±0.4% for the complete recording of all 10 subjects. This shows that embodiments of the model behave well for both FOG and REG events, even if the gaits characteristics differed between individuals. Furthermore, the small standard deviation of all the metrics shows that the model is capable of predicting the FOG events of all patients equally well, despite subjects having different physiological characteristics and different stages of PD.

An embodiment of the algorithm was able to identify the FOG events with high accuracy at a high frequency of 50 Hz using the signals from an embodiment of the DeepSole system. The example implementation uses a window of 0.5 seconds, but generates a prediction at 50 Hz. This property allows the implemented algorithm to identify the episode within one sampling frequency of the embodiment of the DeepSole System, i.e., 20 milliseconds. This was tested with 10 PD patients that suffered from FOG. In an embodiment, the algorithm accurately identified the FOG events with a small number of false predictions without requiring manual calibration, unlike thresholding algorithms. For the implemented model, the ground truth was coded with one second resolution. This was deemed sufficient, as the freezing episodes last longer than this period. This resolution can be improved to better identify the onset of the episode. The combination of an embodiment of the ANN model with an embodiment of the DeepSole System allows a setup that is portable, comfortable to wear, minimally invasive and capable of timely identification of FOG episodes. This represents an improvement as many patients find it challenging to wear systems that require multiple sensors placed on different areas of the body. As shown in Table III, different authors use different numbers of sensors at several segments of the body. Embodiments of the DeepSole system provide a platform where sensors can be contained within a pair of shoes, simplifying both the setup and the comfort.

Some embodiments of the model outperform the previous models in the evaluated metrics. Further studies can focus on the effects of each of the sensors on the network performance. Some embodiments of the model use raw data from 24 sensors placed at the foot of the wearer without any filtering or preprocessing. This was chosen because the ANN in some embodiments can be used as a general regressor between workspaces without requiring engineered features on the data to be found. This is possible due to the ANN's ability to detect possible interactions between the input sensors. Therefore, the sensors can be used without having to create a complex rule set.

By using convolutional and recurrent layers, embodiments of the algorithm can avoid frequency domain transformations like Fast Fourier Transforms. This property contributes to the speed of the prediction being the same as the sampling frequency. To further improve the speed of prediction, different combinations of layers and sensors included in the input could be changed. This would also give insights on the main contributor to the performance of the network.

The dataset collected using some embodiments showed that the gait of PD patients is highly variable. During the stride on the onset of the event, the patients are less stable. With a statistical change to their stride width, toe in/out angle, and center of pressure distance during stance phase. However, further analysis can be done to understand how many strides prior to the events are different to the regular gait. This result also shows that the changes can be different depending on the patient. For example, the study presented by Nieuwboer et al. found differences in the stride length and cadence, but in the dataset from the example implementation these parameters were not statistically different. To minimize the effect of this inter-subject variation, the raw sensor data was used. These signals can capture different spatiotemporal gait characteristics, so the identification should be more robust than with abstract gait parameters.

The wireless capability of embodiments of the DeepSole system allows us to implement an algorithm in real-time at the same sampling frequency as the data acquisition, for example 50 Hz. A simulation was made by sending the real-time session recording to a desktop computer. On a desktop computer with a Nvidia 2080 video card, the model could be run at a frequency of 50 Hz. In a real environment, the identification rate could be affected by unknown variables, like the network latency and loss of data packets. By using real-time identification, auditory or haptic feedback can be provided as needed to the patient. The high frequency detection of 50 Hz could be used to provide sensory feedback to help patients with FOG. This would have a positive impact on their daily living.

Embodiments can track gait cycle with one or more sensors (e.g., pressure sensors and/or accelerometers) such that gait characteristics of a subject's gait cycle can be determined. In addition, embodiments can selectively actuate certain muscles (e.g., leg muscles) using positioned transducer during a targeted portion of the gait cycle, for example to induce a change in the subject's gait. In some observations, it was found that episodes of FOG would be encountered by some PD patients after a change in gait characteristic (e.g., decreased stride width and/or change of balance parameters) was encountered. In some embodiments, a PD patient's gait cycle can be monitored to detect a change in gait characteristic (e.g., decreased stride width and/or change of balance parameters), and a positioned transducer (i.e., vibrator) can be actuated after detection of the changed gait characteristic to mitigate FOG. For example, the transducer can be positioned on the PD patient's leg muscle so that actuation of the transducer can stimulate movement. In some embodiments, the transducer can be positioned to target the rectus femoris, biceps femoris, and/or tibialis anterior, and actuation of the transducer upon or after detection of a changed gait characteristic can mitigate an FOG event.

One aspect of the invention is directed to a third apparatus for detecting freezing of gait (FOG) events for a subject. The third apparatus comprises a first plurality of pressure sensors, each of which is configured to detect a pressure at a respective position beneath the subject's right foot, and a second plurality of pressure sensors, each of which is configured to detect a pressure at a respective position beneath the subject's left foot. The third apparatus also comprises an artificial neural network configured to detect FOG events based on the pressures detected by the first plurality of pressure sensors and the pressures detected by the second plurality of pressure sensors.

Some embodiments of the third apparatus further comprise a transducer configured for positioning against a leg muscle of the subject. The transducer is actuated in response to detection of an FOG event by the artificial neural network. Optionally, in these embodiments, the transducer can comprise a vibrator configured to couple vibration into the leg muscle.

Another aspect of the invention is directed to a third method of detecting freezing of gait (FOG) events for a subject. The third method comprises detecting a pressure at each of a plurality of first positions beneath the subject's right foot, and detecting a pressure at each of a plurality of second positions beneath the subject's left foot. The third method also comprises processing the pressure at each of the plurality of first positions and the pressure at each of the plurality of second positions using an artificial neural network configured to detect FOG events based on the pressure at each of the plurality of first positions and the pressure at each of the plurality of second positions.

Some instances of the third method further comprise actuating a transducer positioned against a leg muscle of the subject in response to detection of an FOG event. Optionally, in these instances, the transducer comprises a vibrator configured to couple vibration into the leg muscle.

In this application, unless specifically stated otherwise, the use of the singular includes the plural and the use of “or” means “and/or.” Furthermore, use of the terms “including” or “having,” as well as other forms, such as “includes,” “included,” “has,” or “had” is not limiting. Any range described herein will be understood to include the endpoints and all values between the endpoints.

Furthermore, the foregoing descriptions apply, in some cases, to examples generated in a laboratory, but these examples can be extended to production techniques. For example, where quantities and techniques apply to the laboratory examples, they should not be understood as limiting. In addition, although specific materials have been disclosed herein, other materials can also be employed according to one or more contemplated embodiments.

Features of the disclosed embodiments can be combined, rearranged, omitted, etc., within the scope of the invention to produce additional embodiments. Furthermore, certain features can sometimes be used to advantage without a corresponding use of other features.

It is thus apparent that there is provided in accordance with the present disclosure, system, methods, and devices for gait analysis and/or training. Many alternatives, modifications, and variations are enabled by the present disclosure. While specific embodiments have been shown and described in detail to illustrate the application of the principles of the disclosure, it will be understood that the disclosure may be embodied otherwise without departing from such principles. Accordingly, alternatives, modifications, equivalents, and variations that are within the spirit and scope of the disclosure are considered in some embodiments.

What is claimed is:

1. An apparatus for modifying a subject's gait, the apparatus comprising:
a plurality of first transducers, each of which is configured for positioning proximate to a respective muscle of a right leg of the subject;
a plurality of second transducers, each of which is configured for positioning proximate to a respective muscle of a left leg of the subject; and
a processor configured to track the subject's gait cycle and, based on the tracked gait cycle, actuate each of the plurality of first transducer and each of the plurality of second transducers at respective portions of the subject's gait cycle over multiple gait cycles.

2. The apparatus of claim 1, wherein the processor is configured so that each of the respective portions is synchronized to a respective time during which a respective corresponding muscle has its highest activation.

3. The apparatus of claim 1, wherein the plurality of first transducers includes a right tibialis anterior transducer, a right rectus femoris transducer, and a right biceps femoris transducer, and
wherein the plurality of second transducers includes a left tibialis anterior transducer, a left rectus femoris transducer, and a left biceps femoris transducer.

4. The apparatus of claim 3, wherein the processor is configured to (a) actuate the right tibialis anterior transducer during a first portion of the gait cycle, starting from right heel strike, (b) actuate the right rectus femoris transducer during a second portion of the gait cycle, (c) actuate the left biceps femoris transducer during a third portion of the gait cycle, (d) actuate the left tibialis anterior transducer during a fourth portion of the gait cycle, (e) actuate the left rectus femoris transducer during a fifth portion of the gait cycle, (f) actuate the right biceps femoris transducer during a sixth portion of the gait cycle.

5. The apparatus of claim 4, wherein the first, second, third, fourth, fifth, and sixth portions of the gait cycle are all non-overlapping.

6. The apparatus of claim 3, wherein the processor is configured to (a) actuate the right tibialis anterior transducer from 0-10% of the gait cycle, starting from right heel strike, (b) actuate the right rectus femoris transducer from 10-30% of the gait cycle, (c) actuate the left biceps femoris transducer from 30-40% of the gait cycle, (d) actuate the left tibialis anterior transducer from 40-60% of the gait cycle, (e) actuate the left rectus femoris transducer from 60-75% of the gait cycle, and (f) actuate the right biceps femoris transducer from 87-100% of the gait cycle.

7. The apparatus of claim 1, wherein each of the first transducers and each of the second transducers comprises a vibrator configured to couple vibration into a respective leg muscle.

8. A method for modifying a subject's gait, the method comprising:
positioning each of a plurality of first transducers proximate to a respective muscle of a right leg of the subject;
positioning each of a plurality of second transducers proximate to a respective muscle of a left leg of the subject;
tracking the subject's gait cycle; and
actuating each of the plurality of first transducer and each of the plurality of second transducers at respective portions of the subject's gait cycle over multiple gait cycles based on the tracked gait cycle.

9. The method of claim 8, wherein each of the respective portions is synchronized to a respective time during which a respective corresponding muscle has its highest activation.

10. The method of claim 8, wherein the plurality of first transducers includes a right tibialis anterior transducer, a right rectus femoris transducer, and a right biceps femoris transducer, and
wherein the plurality of second transducers includes a left tibialis anterior transducer, a left rectus femoris transducer, and a left biceps femoris transducer.

11. The method of claim 10, wherein the actuating comprises (a) actuating the right tibialis anterior transducer during a first portion of the gait cycle, starting from right heel strike, (b) actuating the right rectus femoris transducer during a second portion of the gait cycle, (c) actuating the left biceps femoris transducer during a third portion of the gait cycle, (d) actuating the left tibialis anterior transducer during a fourth portion of the gait cycle, (e) actuating the left rectus femoris transducer during a fifth portion of the gait cycle, (f) actuating the right biceps femoris transducer during a sixth portion of the gait cycle.

12. The method of claim 11, wherein the first, second, third, fourth, fifth, and sixth portions of the gait cycle are all non-overlapping.

13. The method of claim 10, wherein the actuating comprises (a) actuating the right tibialis anterior transducer from 0-10% of the gait cycle, starting from right heel strike, (b) actuating the right rectus femoris transducer from 10-30% of the gait cycle, (c) actuating the left biceps femoris transducer from 30-40% of the gait cycle, (d) actuating the left tibialis anterior transducer from 40-60% of the gait cycle, (e) actuating the left rectus femoris transducer from 60-75% of the gait cycle, and (f) actuating the right biceps femoris transducer from 87-100% of the gait cycle.

14. An apparatus for modifying a subject's gait, the apparatus comprising:
at least two pressure sensors;
a first transducer configured for positioning proximate to a first leg muscle of the subject; and
a processor configured to track the subject's gait cycle responsively to signals from the at least two pressure sensors and, based on the tracked gait cycle, actuate the first transducer to provide feedback to the subject in synchronicity with a first portion of the subject's gait cycle.

15. The apparatus of claim 14, further comprising a second transducer configured for positioning proximate to a second leg muscle of the subject, wherein the processor is configured to actuate the second transducer to provide feedback to the subject in synchronicity with a second portion of the subject's gait cycle.

16. The apparatus of claim 15, wherein the processor is configured to actuate the first transducer in synchronicity with the first portion of the subject's gait cycle and the second transducer in synchronicity with the second portion of the subject's gait cycle over multiple gait cycles.

17. The apparatus of claim 14, wherein the at least two pressure sensors are configured for positioning at the subject's shoe.

18. The apparatus of claim 14, further comprising an accelerometer, wherein the processor is configured to track the subject's gait cycle responsively to signals from the at least two pressure sensors and the accelerometer.

19. The apparatus of claim 14, wherein the processor is configured to actuate the first transducer upon detection of a change in stride characteristic of the tracked gait cycle.

20. The apparatus of claim 14, wherein the processor is configured to actuate the first transducer upon detection of a change in stride length of the tracked gait cycle.
